(19) United States
(12) Patent Application Publication
(10) Pub. No.: US 2024/0374535 A1
(43) Pub. Date: Nov. 14, 2024

(54) COMPOSITIONS AND METHODS FOR TREATING BIOFILM DISORDERS AND INFECTIONS

(71) Applicant: AHV International B.V., Zwolle (NL)

(72) Inventors: Lex DE BOER, Wateringen (NL); Gerrit Jan STREEFLAND, Arriën (NL); Anna Kristina WRONSKA, Braunschweig (DE); Anne Myrthe SCHUTTEVAAR, Hoge Hexel (NL); Shaista Sheroze KHOKHAR, Den Haag (NL)

(21) Appl. No.: 18/690,582

(22) PCT Filed: Sep. 8, 2022
(86) PCT No.: PCT/NL2022/050507

§ 371 Date: Mar. 8, 2024

(30) Foreign Application Priority Data
Sep. 10, 2021 (NL) 2029166

(51) Int. Cl. A61K 31/10; A01N 31/02; A01P 1/00; A61P 17/02; A61P 31/04

(52) U.S. Cl. CPC A61K 31/10; A01N 31/02; A01P 1/00; A61P 17/02; A61P 31/04

(57) ABSTRACT

The disclosure relates to organosulfur containing compositions, in particular Di-n-butyl thiosulfinate, Di-methyl thiosulfonate, Di-phenyl thiosulfonate, Bis (p-tolyl) thiosulfinate, Di-isopropyl thiosulfinate, Di-benzyl thiosulfinate, Di-benzyl thiosulfonate, S-propyl-4-methylbenzene thiosulfonate, Di-n-propyl thiosulfinate, and Di-n-propyl thiosulfonate. Such compositions are useful for treating infection and reducing or degrading biofilms both in vivo and in vitro. In particular, such compositions are useful in the treatment of biofilm-related disorders, including but not limited to mastitis, digital dermatitis, and chronic wound infections.

### FIELD OF THE INVENTION

The disclosure relates to organosulfur containing compositions, in particular Di-n-butyl thiosulfinate, Di-methyl thiosulfonate, Di-phenyl thiosulfonate, Bis (p-tolyl) thiosulfinate, Di-isopropyl thiosulfinate, Di-benzyl thiosulfinate, Di-benzyl thiosulfonate, S-propyl-4-methylbenzene thiosulfonate, Di-n-propyl thiosulfinate, and Di-n-propyl thiosulfonate. Such compositions are useful for treating infection and reducing or degrading biofilms both in vivo and in vitro. In particular, such compositions are useful in the treatment of biofilm-related disorders, including but not limited to mastitis, digital dermatitis, and chronic wound infections.

### BACKGROUND OF THE INVENTION

The health condition of the udder plays an essential role in dairy animals both from a health and wellness perspective as well as from an economic perspective. The infection in mammary glands of dairy animals, such as cows, known as mastitis, has a significant economic impact on dairy farms worldwide. The overall global loss per year is estimated to be four to five billion Euros. In dairy mastitis, the udder is incapable of mounting an efficacious defence response to invading microorganisms. Several factors are known to disrupt the balance at the level of the udder which can compromise the ability of the dairy animal to kill microorganisms causing mastitis. Consequently, host response mechanisms may be incapable of triggering an efficient defence response to eliminate invading pathogens leading to bacterial colonization of the udder and the onset of clinical or subclinical mastitis.

Bacterial colonization and especially the formation of bacterial reservoirs in the udder of dairy cattle are generally difficult to combat, leading to chronic infections that can persist despite certain treatment strategies with or without antibiotics. Important factors associated with the persistence of bacterial infections of the udder in dairy cattle are epithelial adhesion, the production of biofilm and the susceptibility of bacteria to phagocytosis.

Several strategies have been employed in order to control or prevent mastitis in dairy cows, such as antibiotic treatment and vaccination, to name a few, and to reduce the clinical and economic consequences of the disease. However, most treatments and strategies have little or no effect for ameliorating the disease. Several reasons could account for the lack of efficacy. First, although a number of virulence factors have been suggested as potential antigens for single-component vaccines, experimental trials have demonstrated that induction of immunity to single factors is not sufficient to confer robust protection against bacteria causing mastitis in dairy cows. Second, bacterial antigens suffer from low immunogenicity and require adequate adjuvantion. Third, a major challenge in the control of mastitis are effective antibiotics able to reach bacterial pools in the udder for instance as a result of the formation of biofilms by bacteria.

Microorganisms, such as bacteria, do not necessarily need to produce a biofilm, but they have much better possibilities to survive in the host if they can adhere, for instance, to epithelial cells. Adhesion is an active process, involving a series of attachments and detachments, with resulting biofilm formation which is accompanied by significant genetic and subsequent physiological changes in the microorganisms resulting, inter alia, in a loss of sensitivity to virtually all classes of antibiotics. Hence, the management of bacterial udder infections is becoming increasingly difficult due to the emergence and increasing prevalence of bacterial pathogens that are resistant to antibiotics. In some cases, low doses of antibiotics can even enhance biofilm formation suggesting a natural defence mechanism of bacteria in avoiding the lethal effects of antibiotics. Due to this complex and problematic situation, mastitis remains one the most important diseases in dairy cattle despite the progress made in improving general udder health in recent years.

In addition, when cattle are frequently or continuously treated with antibiotics the antibiotics and degradation products are also found in the manure. The manure comprising the antibiotics and degradation products are spread over soil. The presence of the antibiotics is shown to affect the bacterial diversity in the soil. This is an undesirable effect to the environment. An alternative composition to treat infections in cattle will prevent the spread of antibiotics through the environment and recovery of the microbiome of the soil.

Although the description of bacterial biofilms can be found in the scientific literature much earlier, the meaning of biofilms became known in 1982, when Costerton observed that Staphylococcus aureus had formed a biofilm on a cardiac pacemaker lead. Subsequent research and clinical observations revealed that bacterial biofilms can be found on implants and catheters, prosthetic devices and other implanted biomaterials. Even more relevant was the observation that microbial biofilms can be formed also on biological surfaces is human and animal tissues such as the periodontal mucosa in the oral cavity (dental plaque), nasal sinuses (chronic sinusitis), the inner ear (otitis media), blood vessels and heart valves (endocarditis), the alveolar surface (multiple lung diseases) or the biliary and urinary bladder.

The first stage of biofilm formation comprises the attachment of cells to a surface. In the second stage, cell multiplication occurs accompanied by the formation of mature structures have many layers of cells. A slime layer is also formed which further protects the bacteria. See, e.g., Melchior et al. Veterinary Journal 2006 171:398-407. Once a critical mass is reached, the outermost cell layer of a biofilm may release ‘planktonic organisms’. These organisms may further colonize other surfaces. Biofilms may form on a wide variety of surfaces, including living tissues, indwelling medical devices, industrial or potable water system piping, or natural aquatic systems. As will be understood by a skilled person, not all infections lead to the development of biofilms.

Research in the last 20 years revealed that collective biofilm formation is facilitated by bacterial communication system, denoted as quorum sensing (QS). QS occurs by means of small chemical molecules (so called auto-inducers, AI) permanently excreted by bacteria into their environment. These signalling molecules (for example oligopeptides (AIP) or N-acetyl homoserine lactones (AHL)) are recognized and monitored via specific receptors by other bacteria in their neighbourhood. When a specific density of AIs is reached (a quorum), bacterial cells collectively alter gene expression and either produce virulence factors to attack body cells, or to activate metabolic pathways to form a biofilm at tissue surfaces. Biofilm formation involves the formation of an extracellular matrix consisting of large polymers, initially predominantly polysaccharides, which upon maturation are stabilized by proteins and lipids, resulting in three-dimensional structure.

Once a biofilm infection has been established, it can be very difficult to eradicate. Mature biofilms will intermittently release planktonic cells. This can lead to chronic infections with intermittent exacerbations. While antibiotics or the host's immune response may resolve the symptoms due to planktonic cells, the mature biofilms may remain.

Microbial cells under the protection of a biofilm are often more resistant to antibiotics as well as the body's natural immune responses. Dormant bacteria are metabolically inactive and hence do not express the typical targets of many antibiotics such as synthesis of bacterial cell wall constituents (target for beta-lactam antibiotics such as penicillins and cephalosporins and Vancomycin) and rapid protein (target for Aminoglycosides, Tetracyclines, Macrolides and Linezolid) and DNA synthesis (Fluoroquinolones and Rifampicin) or Folic acid synthesis (Sulfonamides, aminopyrimidines (such as Trimethoprim). Therefore, antibiotic treatment alone is generally not sufficient to eradicate biofilm infections (see also Wu et al. Int J Oral Sci. 2015 March; 7(1): 1-7). While antibiotics may be effective against dispersed (planktonic) bacteria, it is difficult to reach within a biofilm the minimal concentration of antibiotic necessary to eradicate the microorganisms within the biofilm. It has been demonstrated in various in vitro experiments that bacteria growing in a biofilm are 10 to 1,000 times more resistant to various antimicrobial agents when compared to the planktonic bacteria of the same strain (see, e.g., Amorena et al. 1999 J. Antimicrob. Chemother. 44:43-55; Ceri et al., 1999 J. Clin. Microbiol. 37:1771-1776; and Olson et al., 2002 Can. J. Vet. Res. 66:86-92). Olsen et al. reviews various mechanisms of biofilm-induced antibiotic resistance and tolerance (Eur J Clin Microbiol Infect Dis 2015 34:877-886).

For bovine mastitis, this has been reported in a study with field strains of Staphylococcus aureus (Melchior, Gaastra & Fink-Gremmels, J. Vet. Med. 2006 53:326-332). The MIC50, Minimum bacterial concentration (BMIC) and MBEC (minimal biofilm eradicating concentration) were determined for 7 strains isolated from mastitis infected cows. Antibiotics tested included those that are commonly used in the treatment of bovine mastitis such as Penicillin, Amoxycillin, Cloxacillin, Cephalothin, Cefoperazone, Cefquinome, Cloxacillin/Penicillin combination, Cloxacillin/Penicillin combination, Lincomycin, Pirlimycin, Tyrosine, Neomycin, Gentamycin, Trimethoprim/Sulfamethoxazole, Florfenicol, and Danofloxacin. For all antibiotics tested the difference between MIC (planktonic bacteria) and MBEC (biofilm eradication concentrations) differed by more than a factor of 256, and very often more than a factor of 2048.

There is also evidence that antibiotics may stimulate biofilm formation. For example, some antibiotics (such as tetracyclines, quinopristine-dalfopristins, and erythromycin) may stimulate the expression of genes (e.g, the ica genes) in bacteria which promotes the adherence of the bacteria (Melchior et al., supra). Interestingly, it has been shown that there is a high prevalence of ica genes among S. aureus mastitis isolates (reviewed in Melchior et al., supra). These results support the hypothesis that mammary infections are associated with biofilm formation.

Biofilms can also comprise dormant bacteria. Biofilms employ a number of mechanisms to evade a host's immune response including activating regulators/suppressors that affect immune cell activity and acting as a physical barrier to immune cells (Gonzalez Pathog Dis. 2018 April; 76(3)), but can transit out of dormancy and become active. In general, the immune system only acts against active bacteria and therefore the dormant bacteria can escape the immune system of an individual. Dormant bacteria are able to detach from the biofilm and quickly become active and harmful to the host.

Fungal related biofilms are also known to be more resistant to antifungal drugs as compared to planktonic cells (see, e.g., Fanning and Mitchell PLOS Pathog 2012 8:e1002585 for a review). Accordingly, compounds having antimicrobial effects are not always suited for the treatment or prevention of biofilms.

In a mature biofilm a dormant stage is adopted by downregulating (gene-shift) of primary metabolism. Dormancy encompasses that biofilm bacteria almost entirely suppress the expression of the typical targets for antibiotics, such as protein and DNA synthesis and cell-wall rebuilding. Subsequently biofilms are inherently insensitive to antibiotics and are often upwards 1000-fold more resistant to them than planktonic (free-floating) bacteria. Furthermore, the higher cell densities found in biofilms considerably increases the probability of horizontal gene transfer, which increases the likelihood of the emergence of strains with increased resistance or altered virulence profiles. The clinical outcome is a phenotypic resistance to common (even modern) antibiotics.

In the protective environment of such a biofilm, bacterial survival time increases.

Moreover, the self-constructed inert polysaccharide-rich matrix is non-immunogenic, protecting the biofilm-embedded bacteria from recognition (via PAMPS—pathogen associated molecular patterns) and engulfment by immune cells of the host.

The formation of biofilms can have serious negative consequences in medical, industrial, and natural settings. In particular biofilm-associated infections (i.e., biofilm related disorders) are a serious problem in both humans and animals. Such disorders may be characterized by a chronic inflammatory response with recurrent acute episodes and resistance to antimicrobial therapy and/or host defenses. Wound biofilms delay tissue repair resulting in chronic wounds. It has been suggested that biofilm infections account now for up to 80% of all human microbial infections (Bartell J A et al., 20 Evolutionary highways to persistent bacterial infection. Nature Communications (2019) 10:629 and Sharma et al. 2019 Antimicrobial Resistance and Infection Control 8:76). Jamal et al. reported that the National Institute of Health indicated that 65% of microbial infections and 80% of chronic infections are associated with biofilm formation (Journal of Chinese Medical Association 81:7-11). Biofilms and biofilm-related disorders have been extensively discussed in the literature; see, e.g., Sharma et al. 2019 Antimicrobial Resistance and Infection Control 8:76; Roy et al. 2018 Virulence 9:522-554; and Jamal et al. 2018 Journal of the Chinese Medical Association 81:7-11. Vestby et al. reviews bacterial biofilms and their role in disease (Antibiotics (Basel). 2020 February; 9(2): 59). A comprehensive overview of biofilm infections was presented in 2014 by the European Clinical Society of Microbiology and Infectious Disease, see also David Lebeaux et al. Microbiol. Mol. Biol. Rev. 2014; doi:10.1128/VIMBR.00013-14.

For example, Pseudomonas aeruginosa, an organism that causes nosocomial infections, forms biofilms on surfaces as diverse as cystic fibrosis lung tissue, contact lenses, and catheter lines. P. aeruginosa growing as biofilms have also been found in chronic wounds and can lead to impaired healing of wounds. Biofilms, in particular, P. aeruginosa biofilms, also cause chronic infections in the respiratory diseases such as bronchiectasis, chronic obstructive pulmonary disease and in chronic rhinosinusitis. Biofilms formed on medical devices serve as a reservoir of bacteria that can be shed into the body, leading to a chronic systemic infection. Candida albicans (a yeast) is the most common fungal biofilm found in hospitals, but is extremely difficult to treat and do not respond well to typical antifungal treatments.

The pioneering studies in bacterial biofilm formation focused on Staphylococcus aureus (Gram-positive) and Pseudomonas aeruginosa (Gram-negative) because of their involvement in recurrent mastitis in dairy cattle and complicated wound infections. In addition, biofilm formation of Streptococcus ssp., avian pathogenic E. coli (APEC) and C. jejuni biofilm gained attention, as these bacterial species are of public health relevance. Subsequently, other important animal pathogens, including Actinobacillus pleuropneumoniae (severe lung infections in swine that might become lethal), Escherichia coli (local and systemic infections and systemic septicaemia, which often is a lethal in poultry), skin and enteric diseases in dogs and cats and wound infections and endometritis in horses were recognized as biofilm infections. This list is non-exclusive as biofilm formation is a general trait of almost all micro-organisms.

Accordingly, a need exists for antimicrobial compounds as well as alternative treatments of biofilm-related disorders.

### SUMMARY OF THE INVENTION

The disclosure provides the following preferred embodiments.
A compound according to Formula I, or a composition comprising a compound according to Formula I:

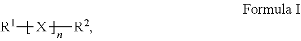

wherein n is 2;
wherein one X is —S— and the other X is selected from the group consisting of —S—, —S(O)—, and —S(O)₂—;
and R¹ and R² are independently selected from optionally substituted alkyl or optionally substituted aryl; wherein the compound according to Formula I is selected from the group consisting of di-n-butyl disulfide, dimethyl disulfide, diethyl disulfide, di-n-propyl disulfide, diethyl thiosulfinate, Di-n-propyl thiosulfinate, Di-n-butyl thiosulfinate, Di-n-propyl thiosulfonate, Di-methyl thiosulfonate, and Di-phenyl thiosulfonate; for use in the treatment of a biofilm-related disorder.
A compound according to Formula I, or a composition comprising a compound according to Formula I:

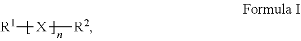

wherein n is 2;
wherein one X is —S— and the other X is selected from the group consisting of —S—, —S(O)—, and —S(O)₂—;
and R¹ and R² are independently selected from optionally substituted alkyl or optionally substituted aryl;
for use in the treatment of a biofilm-related disorder.
The compound or composition for use according to any one of the preceding embodiments, wherein the biofilm-related disorder is a chronic and/or persistent infection.
The compound or composition for use according to any one of the preceding embodiments, wherein the biofilm-related disorder is digital dermatitis, udder cleft, or a chronic wound infection.
The compound or composition for use according to any one of the preceding embodiments, wherein the biofilm comprises bacteria, yeast, fungi, microalgae, or a combination thereof.
A compound according to Formula I, or a composition comprising a compound according to Formula I:

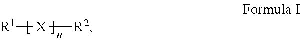

wherein n is 2;
wherein one X is —S— and the other X is selected from the group consisting of —S—, —S(O)—, and —S(O)₂—;
and R¹ and R² are independently selected from optionally substituted alkyl or optionally substituted aryl;
for use in the treatment or prevention of a microbial infection, preferably a bacterial or fungal infection.
The compound or composition for use according to any one of the preceding embodiments, wherein the use further comprises the administration of an antimicrobial agent, preferable selected from an antifungal or an antibiotic.
The compound or composition for use according to any one of the preceding embodiments, wherein the use further comprises the administration of an anti-inflammatory agent.
A composition comprising a compound according to Formula I:

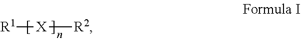

wherein n is 2;
wherein one X is —S— and the other X is selected from the group consisting of —S—, —S(O)—, and —S(O)₂—;
and R¹ and R² are independently selected from optionally substituted alkyl or optionally substituted aryl;
wherein the composition is a pharmaceutical composition, functional food, or a cleaning product.
An in vitro method for preventing or reducing biofilm formation or growth on a surface or for degrading or reducing biofilms on a surface,
said method comprising applying a composition to the surface such as to prevent or reduce biofilm formation or growth on a surface,
or such as to degrade or reduce biofilms on a surface, wherein said composition comprises a compound according to Formula I:

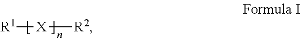

wherein n is 2;
wherein one X is —S— and the other X is selected from the group consisting of —S—, —S(O)—, and —S(O)₂—;
and R¹ and R² are independently selected from optionally substituted alkyl or optionally substituted aryl.
The compound or composition for use, the method, or the composition according to any one of the preceding embodiments, wherein the composition is essentially free of diallyl thiosulfinate.
The compound or composition for use, the method, or the composition according to any one of the preceding embodiments, wherein the composition further comprises an antimicrobial agent, preferable selected from an antifungal or an antibiotic.
The compound or composition for use, the method, or the composition according to any one of the preceding embodiments, wherein the composition further comprises anti-inflammatory agent.
An article having a surface at least partially coated with a compound according to Formula I:

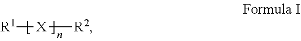

wherein n is 2;
wherein one X is —S— and the other X is selected from the group consisting of —S—, —S(O)—, and —S(O)₂—;
and R¹ and R² are independently selected from optionally substituted alkyl or optionally substituted aryl; preferably, wherein the article is a medical device or surgical device.
The compound or composition for use, the method, the article, or the composition according to any one of the embodiments, wherein R¹ and R² are independently selected from C₁₋₆ alkyl, and phenyl; preferably R¹ and R² are independently C₁₋₄ alkyl.
The compound or composition for use, the method, the article, or the composition according to any one of the embodiments, wherein R¹ and R² are identical.
The compound or composition for use, the method, the article, or the composition according to any one of the embodiments, wherein, each X is —S—, and R¹ and R² are independently C₁₋₄ alkyl.
The compound or composition for use, the method, the article, or the composition according to any one of the embodiments, wherein one X is —S— and the other X is selected from the group consisting of —S(O)— and —S(O)₂—; and R¹ and R² are independently selected from alkyl, aryl, alkylaryl, and arylalkyl.
The compound or composition for use, the method, the article, or the composition according to any one of the embodiments, wherein one X is —S— and when the other X is —S(O)—, R¹ and R² are independently selected from the group consisting of n-propyl, isopropyl, butyl, benzyl, and p-tolyl; and/or
wherein when the other X is —S(O)₂—, R¹ and R² are independently selected from the group consisting of methyl, phenyl, benzyl, 4-methylbenzene, and n-propyl.

Preferably, wherein one X is —S— and when the other X is —S(O)—, R¹ and R² are independently selected from the group consisting of isopropyl, butyl, benzyl, and p-tolyl; and
wherein when the other X is —S(O)₂—, R¹ and R² are independently selected from the group consisting of methyl, phenyl, benzyl, 4-methylbenzene, and n-propyl.
The compound or composition for use, the method, the article, or the composition according to any one of the embodiments, wherein the compound according to Formula I is selected from the group consisting of dimethyl disulfide, diethyl disulfide, di-n-propyl disulfide, di-n-butyl disulfide, diethyl thiosulfinate, Di-n-propyl thiosulfinate, Di-n-butyl thiosulfinate, Di-n-propyl thiosulfonate, Di-methyl thiosulfonate, and Di-phenyl thiosulfonate.
The compound or composition for use, the method, the article, or the composition according to any one of the embodiments, wherein the compound according to Formula I is selected from the group consisting of Di-n-butyl thiosulfinate, Di-methyl thiosulfonate, Di-phenyl thiosulfonate, Bis (p-tolyl) thiosulfinate, S-propyl-4-methylbenzene thiosulfonate, Di-isopropyl thiosulfinate, Di-benzyl thiosulfinate, Di-benzyl thiosulfonate, Di-n-propyl thiosulfinate, and Di-n-propyl thiosulfonate.
The compound or composition for use, the method, the article, or the composition according to any one of the embodiments, wherein the compound according to Formula I is selected from the group consisting of Di-n-butyl thiosulfinate, Di-methyl thiosulfonate, and Di-phenyl thiosulfonate.

Method of treatments are also disclosed comprising administering a compound of Formula I to an individual in need thereof. Such methods are useful for treating a biofilm disorder as disclosed herein. Such methods are also useful for treating infections as disclosed herein.

### BRIEF DESCRIPTION OF THE DRAWINGS

FIG. 1 Somatic cell counts of cows during 60 days after treatment.

### DETAILED DESCRIPTION OF THE DISCLOSED EMBODIMENTS

The disclosure relates to compounds according to Formula I:

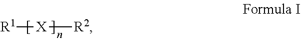

wherein n is 2;
wherein one X is —S— and the other X is selected from the group consisting of —S—, —S(O)—, and —S(O)₂—;
and R¹ and R² are independently selected from optionally substituted alkyl and optionally substituted aryl. Such compounds (also referred to herein as organosulfur compounds) are useful for reducing, degrading, and/or preventing the formation of biofilms.

As used herein, “alkyl” relates to a saturated aliphatic hydrocarbyl group. Unless stated otherwise, an alkyl group can be linear or branched. Preferably, alkyl groups are linear. As used herein, alkyl groups can be substituted or unsubstituted. Preferably, alkyl groups are unsubstituted. In preferred embodiments, in Formula I an alkyl is a C₁₋₆ alkyl, more preferably a C₁₋₄ alkyl.

As used herein, “aryl” refers to an aromatic hydrocarbon ring system that comprises six to twenty-four carbon atoms, more preferably six to twelve carbon atoms, and may include monocyclic and polycyclic structures. When the aryl group is a polycyclic structure, it is preferably a bicyclic structure. Optionally, the aryl group is substituted by one or more substituents further specified in this document. Preferably, an aryl group is unsubstituted. Examples of aryl groups are phenyl, benzyl, and naphthyl. Most preferably, an aryl group is phenyl.

As used herein, “substituted” indicates that a group contains one or more substituents. Preferably, the substituents are independently selected from the group consisting of halogen, —C(O)OH, —C(O)NH₂, —OH, ═O, C₁₋₃ alkoxy, —NH₂, —NO₂, —SO₃H, and CF₃. Preferably, halogens are selected from the group consisting of —Cl, —F, —Br, and —I. Most preferably, a halogen is —Cl. In preferred embodiments, the groups as disclosed herein contain at most three substituents, more preferably at most two substituents, and most preferably at most one substituent.

In preferred embodiments, R¹ and R² are independently selected from alkyl and aryl. More preferably, R¹ and R² are independently selected from C₁₋₆ alkyl, and phenyl. Even more preferably, R¹ and R² are independently C₁₋₄ alkyl. Most preferably, R¹ and R² are independently linear C₁₋₄ alkyl, i.e. methyl, ethyl, n-propyl, or n-butyl.

In preferred embodiments, R¹ and R² are identical.

In preferred embodiments, each X is —S—. In preferred embodiments, one X is —S— and the other X is —S(O)—. In other preferred embodiments, one X is —S— and the other X is —S(O)₂—.

In preferred embodiments, in Formula I each X is —S—, and R¹ and R² are independently C₁₋₄ alkyl, preferably linear C₁₋₄ alkyl, i.e. methyl, ethyl, n-propyl, or n-butyl. Preferably, compositions comprising these particular compounds according to Formula I further comprise one or more antimicrobial agents.

In preferred embodiments of Formula I, one X is —S— and the other X is selected from the group consisting of —S(O)— and —S(O)₂— and R¹ and R² are independently selected from alkyl, aryl, alkylaryl, and arylalkyl.

Preferably, wherein one X is —S— and when the other X is —S(O)—, R¹ and R² are independently selected from the group consisting of n-propyl, isopropyl, butyl, benzyl, and p-tolyl; and/or wherein when the other X is —S(O)₂—, R¹ and R² are independently selected from the group consisting of methyl, phenyl, benzyl, 4-methylbenzene, and n-propyl.

In preferred embodiments, the compound of Formula I is not propyl-propane thiosulfonate (PTSO, also referred to as Di-n-propyl thiosulfonate) or propyl-propane-thiosufinate (PTS, also referred to as Di-n-propyl thiosulfinate).

Preferably, wherein one X is —S— and when the other X is —S(O)—, R¹ and R² are independently selected from the group consisting of isopropyl, butyl, benzyl, and p-tolyl; and wherein when the other X is —S(O)₂—, R¹ and R² are independently selected from the group consisting of methyl, phenyl, benzyl, 4-methylbenzene, and n-propyl.

In preferred embodiments, the compound according to Formula I is selected from the group consisting of dimethyl disulfide, diethyl disulfide, di-n-propyl disulfide, di-n-butyl disulfide, diethyl thiosulfinate, n-propyl propane-1-thiosulfinate (also referred to as Di-n-propyl thiosulfinate), n-butyl butane thiosulfinate (also referred to as Di-n-butyl thiosulfinate), n-propyl propane-1-thiosulfonate (also referred to as Di-n-propyl thiosulfonate), methyl methane thiosulfonate (also referred to as Di-methyl thiosulfonate), and phenyl benzene thiosulfonate (also referred to as Di-phenyl thiosulfonate). In preferred embodiments, the compound according to Formula I is selected from the group consisting of dimethyl disulfide, diethyl disulfide, di-n-propyl disulfide, and di-n-butyl disulphide. In preferred embodiments, the compound according to Formula I is selected from the group consisting of diethyl thiosulfinate, Di-n-propyl thiosulfinate, and Di-n-butyl thiosulfinate. In preferred embodiments, the compound according to Formula I is selected from the group consisting of Di-n-propyl thiosulfonate, Di-methyl thiosulfonate, and Di-phenyl thiosulfonate. In preferred embodiments, the compound according to Formula I is selected from the group consisting of Di-n-butyl thiosulfinate, Di-methyl thiosulfonate, Di-phenyl thiosulfonate, Bis (p-tolyl) thiosulfinate, S-propyl-4-methylbenzene thiosulfonate, Di-isopropyl thiosulfinate, Di-benzyl thiosulfinate, and Di-benzyl thiosulfonate.

In preferred embodiments, the compound according to Formula I is dimethyl disulphide. In preferred embodiments, the compound according to Formula I is diethyl sulphide. In preferred embodiments, the compound according to Formula I is di-n-propyl disulfide. In preferred embodiments, the compound according to Formula I is di-n-butyl disulfide. In preferred embodiments, the compound according to Formula I is diphenyl disulfide. In preferred embodiments, the compound according to Formula I is diethyl thiosulfinate. In preferred embodiments, the compound according to Formula I is Di-n-propyl thiosulfinate. In preferred embodiments, the compound according to Formula I is n-butyl butane thiosulfinate. In preferred embodiments, the compound according to Formula I is Di-n-propyl thiosulfonate. In preferred embodiments, the compound according to Formula I is Di-methyl thiosulfonate. In preferred embodiments, the compound according to Formula I is Di-phenyl thiosulfonate.

It will be understood that the terms “sulphide” and “sulfide” are used interchangeably herein.

In some embodiments, the compounds are obtained from natural sources such as plants. Compounds can be extracted from plant material in various ways. The appropriate method depends on the chemical properties of the compounds. For example, the extraction can start with a non-polar solvent and follow that with solvents of increasing polarity. The compounds are also commercially available or can be prepared as described in example 1.

The disclosure provides compositions comprising the compounds as disclosed herein. Preferably, such compositions are substantially free of diallyl thiosulfinate (also referred to as allyl-2-propene-1-sulfinothioate). Diallyl thiosulfinate is better known under the name Allicin. Allicin is an organosulfur compound. When fresh garlic is chopped or crushed, the enzyme alliinase converts alliin into allicin, which is responsible for the aroma of fresh garlic. The allicin generated is unstable and quickly changes into a series of other sulfur-containing compounds such as diallyl disulfide.

As used herein, “substantially free” refers to compositions comprising less than 5 wt % of diallyl thiosulfinate. In some embodiments the compositions comprise less than 1 wt % of diallyl thiosulfinate, preferably less than 0.5 wt % diallyl thiosulfinate. In some embodiments, the composition comprises a ratio of a compound having formula I to diallyl thiosulfinate by weight of at least 10:1, more preferably of at least 100:1.

The compositions of the present disclosure are also preferably substantially free of diallyl-disulfide. In some embodiments the compositions comprise less than 1 wt % of diallyl disulfide, preferably less than 0.5 wt % diallyl disulfide. In some embodiments, the composition comprises a ratio of a compound having formula I to diallyl-disulfide by weight of at least 10:1, more preferably of at least 100:1.

In some embodiments, compositions are provided wherein at least 50%, preferably at least 90% by weight of the active ingredients are compounds according to formula I as disclosed herein. In some embodiments, compositions are provided wherein the only active ingredients are compounds according to formula I, optionally including further antimicrobial agents and/or anti-inflammatory agents. In some embodiments, compositions are provided wherein at least 50%, preferably at least 90% by weight of the active ingredients are compounds according to formula I as disclosed herein. In some embodiments, compositions are provided wherein the only active ingredients are compounds according to formula I, optionally including further antimicrobial agents and/or anti-inflammatory agents.

The compounds disclosed herein and compositions comprising same are useful in the treatment or prevention of infection. For example, particular uses are for the treatment or prevention of respiratory infection, bowel infection, breast infection, udder infection, skin infection, bladder infection, ear infection, systemic infection, joint infection, brain infection.

As used herein, “infection” refers to, e.g., pathogenic infections which can lead to disease. In particular, such infections are bacterial or fungal infections. Preferably, the infection is a microbial infection. In a preferred embodiment, the infection is a bacterial infection. In a preferred embodiment, the infection is a fungal infection (including yeast infection).

Bacteria and fungi are found almost everywhere and exist in very diverse forms. Most are not harmful and are actually indispensable for life on earth and essential for plant, animal and human health. For example, the microbiome in the intestines of humans and animals where bacteria and fungi live as symbionts with their host is the so-called gut flora. Also, bacteria are naturally present on the skin, which form part of the immune system. Another example is the soil biology, which for the most part consists of bacteria and fungi. Some bacteria and fungi can cause pathogenic infections, for example in animals, or humans. These pathological infections can lead to disease and illness of the infected individual.

As used herein, “treatment of infection” refers to a reduction in the severity and/or duration of the infection and/or a reduction of the severity and/or duration of symptoms from the infection. Preferably, said treatment results in restoration of the health of an individual. Preferably, the individual has less disease symptoms or for a shorter time. As used herein, “prevention of infection” refers to the prevention of or alternatively delaying the onset of infection or of one or more symptoms associated with infection.

Some microorganisms, such as bacteria, microalgae, fungi, etc., can form biofilms. The compounds disclosed herein are also useful in the prevention or reduction of biofilm formation or growth and/or for degradation or reduction of biofilms. Preferably, the compounds, and compositions comprising same, are useful in the treatment or prevention of biofilm-related disorders. As a skilled person will recognize, not all compounds which are able to treat microbial (e.g., bacterial) infections are capable of treating biofilm-related disorders.

The term biofilm was initially used in technical and environmental microbiology to describe a community of sessile bacteria and other microorganisms attached to natural or artificial surfaces. The formation of a microbial biofilms is initiated by the colonization of bacteria on a surface to which they attach and produce a slimy film consisting of organic polymers. This primary bacterial film attracts other microorganisms such as algae and protozoa, fungi and protozoa resulting in the formation of a visible multispecies biofilm. Such three-dimensional biofilms are ubiquitous in nature and found on all surfaces that are in contact with water. Of public health concern are microbial biofilms in municipal water supplies and household water pipelines and devices.

As used herein, the term “biofilm” refers to a population of microorganisms that are concentrated at an interface (usually solid/liquid) and typically surrounded by an extracellular polymeric slime matrix. Biofilms may form on living or non-living surfaces and are found in natural, industrial and hospital settings. Biofilms can contain many different types of micro-organisms, e.g. bacteria, archaea, protozoa, fungi and algae. Preferably, such biofilms comprise bacteria, microalgae (such as Prototheca spp.) or fungi.

As used herein, “treatment of a biofilm-related disorder”, also referred to herein as a “biofilm associated disorder”, refers to a reduction in the severity and/or duration of the disorder and/or a reduction of the severity and/or duration of symptoms from the disorder, in particular the symptoms of infection. Preferably, said treatment results in restoration of the health of an individual. Preferably, the individual has less disease symptoms or for a shorter time.

As used herein, “prevention or reduction of biofilm formation or growth” refers to the prevention, delay, or reduction of biofilm formation or growth. As will be understood by a skilled person, such reduction of biofilm formation or growth may slow the growth of biofilms as compared to the growth of untreated biofilms. Preferably, the compositions are useful for reducing biofilm formation or growth. As used herein, “degradation or reduction of biofilms” refers to the elimination, either partially or completely, of a biofilm. As will be understood by a skilled person, after such treatment, planktonic bacteria may still be present.

The compounds disclosed herein may be capable of disrupting the structure of the biofilm, for example the extracellular mucous matrix. In some embodiments, the compounds are useful for inhibiting cell adhesion. In particular, the compounds may prevent the adhesion (without killing bacteria) to a static or live surface of all cell types encountered in microbial biofilms in particular free living microbes.

While not wishing to be bound by theory, the present disclosure proposes that the therapeutic compounds disclosed herein may exert part of their effects by affecting quorum sensing. Quorum Sensing (QS) signalling plays an important role in the control of e.g. the expression of bacterial virulence factors. QS involves the accumulation of signalling molecules in the surrounding environment which enables a single cell to sense the density of the number of bacteria and the signalling molecules, and therefore the bacterial population as a whole, can make a coordinated response. These cell-cell communication systems regulate various functions of the bacteria such as motility, virulence, sporulation, antibiotic production, DNA exchange, and development of more complex multicellular structures such as biofilm. Therefore, the interference with QS signalling systems might offer a new strategy to combat persisting (chronic) bacterial infections. The ability of certain substances, such as naturally occurring compounds that have Quorum Quenching (QQ) abilities can be used as anti-adhesive compounds and as compounds that interfere with biofilm formation.

The compounds disclosed herein are particularly useful for treating biofilm-related disorders, wherein the disorder is characterized by a chronic and/or persistent infection. The compounds disclosed herein are particularly useful for the treatment of a chronic and/or persistent infection. The terms persistent infection and chronic infection are often used interchangeably, but are based on different mechanisms. Persistent infections are normally held in check by immune defenses but may be activated when such immune defenses are weakened. Persistent infections are often asymptomatic and become clinically visible only when the immune defense fails to control the pathogen. Although persistent infections are often asymptomatic, a skilled person is well aware of means to detect such persistent infections, including e.g., detecting microorganisms from patient samples (e.g., blood or urine). In a chronic infection, pathogens remain in a group of cells/parts of tissue (e.g., joints or lung tissue). The patient always has symptoms of disease, although these might be milder that in the acute phase of infection.

A prominent example of a biofilm disease is bovine mastitis. Bovine mastitis is the clinical term for infections of the mammary gland of cows and can be caused by multiple pathogens, the most prevalent forms are Staphylococcus aureaus, Streptococcus uberis, Streptococcus agalactia, Streptococcus dysgalactiae as well as Serratia marescens and other facultative pathogenic Enterobacteriaceae and Prototheca spp, the latter considered as an emerging pathogen causing an aggressive, non-curable mastitis in many regions of the world. The invention encompasses treating biofilms comprising such microorganisms with the compounds and compositions disclosed herein.

In some embodiments, the wounds treated by the compound of the invention comprise, for example, Staphylococcus aureus; Streptococci; gram negative bacteria, for example Treponema spp., Escherichia coli, Yersiania pestis, Pseudomonas aeruginosa; or yeast/fungi, for example Candida spp (albicans), Cladosporidium herbarum, Trichosporum, Rhodosporidium, and Malassezia.

It is now generally recognized that biofilm-associated microorganisms cause a large number of infections including endocarditis, osteomyelitis, sinusitis, urinary tract infections, chronic prostatitis, periodontitis, chronic lung infection in cystic fibrosis patients, middle ear infections, and various nosocomial infections, especially those related to all known indwelling devices (catheters, implants). The burden of biofilm-disease is significant and represents a major concern in medical care.

While almost all bacterial species are now known to be able to form biofilms under conditions of stress, in current clinical practice several bacterial and fungal species are of major interest, as the associated infections are almost entirely therapy-resistant even if the non-biofilm, planktonic form of the same species and strains show a very good sensitivity to common therapeutic agents (antibiotic and antimycotics/fungicidal agents).

Examples of pathogens which are of major clinical concern due to their biofilm associated therapy-resistance are listed below:
Aspergillus fumigatus—Lung aspergillosis (fungal disease)
Burkholderia cepacian—pulmonary cystic fibrosis superinfections
Candida spp (Yeast)—mucosal surfaces of the gastro-intestinal and urogenital tract
Gardnerella vaginalis (urogenital tract)
Escherichia coli (multiple organs and septicaemia)
Pseudomonas aeruginosa (multiple organs and lung infections including cystic fibrosis)
Staphylococcus aureus (multiple tissues and wound infections, nosocomial infections)
Staphylococcus epidermidis (multiple tissues and wound infections)
Stenotrophomonas maltophilia (chronic respiratory tract diseases).

The treatment of such biofilm-related disorders and biofilm-associated microorganisms is encompassed by the invention. In a preferred embodiment, the biofilm related disorder is udder cleft. Other include which may be treated with a compound of formula I include ulcers (e.g., diabetic foot ulcers (DFUs), venous leg ulcers (VLUs), and pressure ulcers (PUs)); mortellaro (digital dermatitis); and eczema.

Microbial biofilms are not only formed by bacteria, but also by other microorganisms, particularly pathogenic fungi (Aspergillus fumigatus a major cause of multiple Aspergillus-related lung diseases) and yeasts (Candida spp) colonizing of mucosal surfaces of the gastro-intestinal and uro-genital tract. The most prevalent implant-related biofilms are formed by Staphylococcus aureus (MSSA and MRSA), Candida albicans, Pseudomonas aeruginosa, Klebsiella pneumonia, and Enterococcus faecalis. Additionally, microalgae such as, e.g., Prototheca spp can form biofilms and are a source of disease in humans and animals (Protothecosis).

In some embodiments, the biofilm comprises bacteria selected from one or more of Treponema spp, Yersiania pestis, Staphylococcus aureus, Streptococcus agalactiae, Streptococcus dysgalactiae, Streptococcus uberis, Serratia marescens, Trueperella pyogenes, Mannheimia haemolytica, Pasteurella multocida, Pseudomonas aeruginosa, Burkolderia cepacia, Streptococcus neumoniae, Hemophilus influenza, Legionella neumophila, Fusobacterium necrophorum, Corynebacterium pseudotuberculosis, Streptococcus spp., Porphyromonas gingivalis, Pseudomonas aeruginosa, Enterococcus faecalis, Neisseria gonorrhoeae, Escherichia coli, Salmonella enteritidis and Pseudomonas aeruginosa. In some embodiments, the biofilm comprises fungi selected from Absidia spp., Actinomyces spp., Aspergillus spp., Botrytis spp., Candida spp., Centrospora spp., Cephalosporium spp., Ceratocystis spp., Chaetoconidium spp., Chaetomium spp., Cladosporium spp., Colletotrichum spp, Conidiobolus spp., Fulvia spp., Fusarium spp., Geotrichum spp., Guignardia spp., Helminthosporium spp., Histoplasma spp., Lecythophora spp., Malassezia spp., Nectria spp., Nocardia spp., Oospora spp., Ophiobolus spp., Paecilomyces spp., Paracoccidioides brasiliensis, Penicillium spp Phymatotrichum spp., Phytophthora spp., Pythium spp., Piedraia hortai, Rhizoctonia spp., Rhizopus spp., Rhodosporidium spp. Saccharomyces spp., Scerotium spp., Sclerotinia spp., Torulopsosis spp., and Trichophyton spp. Many medically important fungi, such as Candida, Aspergillus, Cryptococcus, Trichosporon, Coccidioides, and Pneumocystis, are known to produce biofilms. In some embodiments the biofilm comprises microalgae, e.g., Prototheca spp.

In certain embodiments, the bacterial infection or biofilm associated disorder is caused by a Gram-negative bacterium. In certain embodiments, the bacterial infection or biofilm associated disorder is caused by a Gram-positive bacterium. In certain embodiments, the bacterial infection or biofilm associated disorder is caused by a multidrug-resistant bacterium. In certain embodiments, the bacterial infection is a methicillin-resistant Staphylococcus aureus (MRSA)-related infection or a Staphylococcus epidermidis (e.g., MRSE) related infection.

In a preferred embodiment the biofilm causing bacteria is Escherichia coli, preferably the biofilm infection is recurrent urinary tract infection, catheter-associated urinary tract infection, or biliary tract infection.

In a preferred embodiment, the biofilm causing bacteria is Pseudomonas aeruginosa, preferably the biofilm infection is Cystic fibrosis lung infection, chronic wound infection, catheter-associated urinary tract infection, chronic rhinosinusitis, chronic otitis media, bronchiectasis, chronic obstructive pulmonary disease or contact lens-related keratitis.

In a preferred embodiment, the biofilm causing bacteria is Staphylococcus aureus, preferably the biofilm infection is Chronic osteomyelitis, chronic rhinosinusitis, endocarditis, chronic otitis media, or of (orthopaedic) implants.

In a preferred embodiment, the biofilm causing bacteria is Staphylococcus epidermidis, preferably the biofilm infection is Central venous catheter, orthopaedic implants, or chronic osteomyelitis.

In a preferred embodiment, the biofilm causing bacteria is Streptococcus pneumoniae, preferably the biofilm infection is infection of nasopharynx, chronic rhinosinositis, chronic otitis media, or infection in chronic obstructive pulmonary disease.

In a preferred embodiment, the biofilm causing bacteria is Streptococcus pyogenes, preferably the biofilm infection is infection of oral cavity and nasopharynx, recurrent tonsilitis.

Clinical signs of biofilm infections are known to the medial practitioner (see, e.g., Table 1 of Wu et al. Int J Oral Sci. 2015 March; 7(1): 1-7). Such biofilm disorders may lead to chronic infections. The determination of acute versus chronic infection is also known to the practitioner. For example, according to the Mayo Clinic, the occurrence of a yeast infection 4 or more times within a year indicates the presence of a chronic yeast infection whereas the occurrence of two or more bladder infections during a 6-month period indicates the presence of a chronic bladder infection (also referred to as recurrent urinary tract infection).

The most common method to treat a pathological infection of bacteria is the use of antibiotics. Current antibiotics operate primarily through growth-dependent mechanisms and target rapidly-dividing bacteria. However, non-replicative or slower growing bacteria (e.g., dormant persister cells, biofilms) display high levels of antibiotic tolerance and/or resistance contributing to persistent and recurring infection. The compounds disclosed herein are suitable for the use in infections or biofilms comprising antibiotic resistant bacteria, antibiotic tolerant bacteria, and antibiotic persistent bacteria. The compounds disclosed herein are also suitable as a second-line therapy, or rather for individuals that did not response to previous treatment (e.g., antimicrobial treatment) or the disorder returned within e.g., one year or 6 months.

The disclosure further provides the compounds as disclosed herein and compositions comprising same for treating any disorder induced by or relating to biofilms. Disorders induced by or relating to biofilms are well-known to a skilled person. In particular, such disorders are biofilm-related infections. Suitable disorders for treatment include, for example, bacterial prostatitis, bacterial vaginosis, biliary tract infections, chronic sinusitis, chronic lung disease, dental caries, endocarditis, kidney stones, laryngitis, lung infection in cystic fibrosis, gingivitis mastitis, middle ear infections, nonsocomial (bloodstream) infections, obstructive pulmonary diseases, osteomyelitis, otitis media, periodontitis, pneumonia prostatitis, rhinosinusitis, sinusitis, tonsillitis, tuberculosis, urinary tract infections, and wound infections. For example, Mycoplasma Bovis is known to cause udder infection and Joint infection. Biofilm-related disorders also include disorders caused by biofilms formed on indwelling devices (e.g., medical implants, catheters, etc.). Generally, such disorders are treated by removing/replacing the implant. In a preferred embodiment the disorder is mastitis. In some embodiments, the disorder is not mastitis. In some embodiments the treatment is not for inflammatory bowel disease and in particular is not colitis.

In some embodiments, the compounds and compositions as disclosed herein are also useful for treating and preventing infections of implanted medical devices such as joint prosthesis and heart valves as disclosed further herein.

In some embodiments, the compounds and compositions as disclosed herein are also useful for preventing or reducing inflammation in response to bacterial infection or biofilms. Inflammation is part of the complex biological response of body tissues to harmful stimuli, such as pathogens, and is a protective response involving immune cells and molecular mediators. A function of inflammation is to eliminate the pathogens.

In a preferred embodiment, treatment of an individual with the compounds as disclosed herein or compositions comprising same prevents or reduces a clinical inflammation in an animal, e.g., a cow. Preferably the treatment prevents or reduces a (clinical) inflammation of the udder. In another embodiment, treatment of an individual with the compounds and compositions comprising same prevents or reduces a (clinical) inflammation in a human. For example, the treatment prevents or reduces inflammation of the skin, preferably prevents eczema.

While not wishing to be bound by theory, treatment of an individual with the compounds as disclosed herein, or compositions comprising same, reduces biofilm formation or growth and/or causes degradation or reduction of biofilms. Therefore, the individual no longer responds with an inflammatory reaction in the presence of the pathogens. With other words, clearance of the biofilms and pathogens within these biofilms reduces the inflammation response and prevents a clinical inflammation.

During treatment of a biofilm, the microorganisms (such as bacteria and fungi) are released from the biofilm. In some cases, the individual's immune system will respond to the active microorganism. This may result in inflammation of the tissue. The activated immune cells and the inflammatory response can also damage the tissue, for example in the milk gland. Suppression of the inflammatory response may therefore prevent or reduce damage to the tissue.

Furthermore, after tissue has been damaged and the inflammation has ended, the body starts the repair. The macrophages still present stimulate the production of new blood vessels. They also ensure the attraction of fibroblasts. These fibroblasts ultimately cause the formation of granulation tissue. For example, in the case of treatment of a dairy cow, scar tissue may be formed instead of milk producing tissue. The milk production of the cow may therefore be lower than before the inflammation.

In addition to the compounds described herein, further anti-inflammatory drugs can be administered to suppress the inflammatory response and reduce the tissue damage. In a preferred embodiment, the treatments disclosed herein (both therapeutic and prophylactic) further comprise the administration of an anti-inflammatory agent. Anti-inflammatory agents include, for example, nonsteroidal anti-inflammatory agents (cox/lox inhibitors) such as ibuprofen, paracetamol, aspirin, diclofenac, ketoprofen, tolmetin, etodolac, and fenoprofen. Natural anti-inflammatory agents such as Curcumin, Ginger, Spirulina, Cayenne, Cinnamon, Clove, Sage, Rosemary, Black Pepper, natural aspirins, Boswelia, Sanguinaria, and/or Green Tea may also be used. In some embodiments, the methods and uses disclosed herein comprise the combined treatment of the therapeutic organosulfur compounds disclosed herein with an anti-inflammatory agent. The compounds may be administered together or separately. In some embodiments, compositions are provided comprising the therapeutic organosulfur compounds disclosed herein with an anti-inflammatory agent.

In some embodiments, the methods comprise administering to an individual in need thereof compositions comprising the compounds disclosed herein, preferably such as to prevent or reduce biofilm formation or growth, degrade or reduce biofilms, and/or treat or prevent infection (in particular bacterial or fungal infection). In some embodiments, the composition can be administered to an individual for the treatment (e.g., therapeutic agent) or prevention (e.g., prophylactic agent) of a disease or disorder or infection. In some embodiments the individual has or is at risk of developing a biofilm-related infection.

The compositions can be administered to any individual, in particular to animals. Preferable, the animal is a ruminant (such as cows and goats), more preferably a cow. In some embodiments, the animal is not a cow. Preferably, the animal is a non-ruminant, such as a monogastric, a rodent, non-human primate, porcine, equine, canine, feline, or avian. In preferred embodiments the animal is a human. In some embodiments the animal is a non-human animal. In some embodiments, the animal is not an aquatic animal such as fish, mollusks, and crustaceans. Preferably, the animal is a mammal or bird.

While not wishing to be bound by theory, the disclosure provides that the compositions disclosed herein can have advantageous effects after a single administration. In a preferred embodiment, effects are achieved by providing a single oral administration of the composition disclosed herein. Such oral dosing may be, e.g., as a tablet which provides an extended release of the compounds disclosed herein.

The disclosure also provides for multiple administrations. For example, the compositions may be provided more than once per day, daily, weekly, or monthly. In an exemplary embodiment the composition may be provided once daily for a week or until symptoms are alleviated.

Actual dosage levels of the pharmaceutical preparations described herein may be varied so as to obtain an amount of the active ingredient which is effective to achieve the desired therapeutic response for a particular patient, composition, and mode of administration, without being toxic to the patient. The selected dosage level will depend upon a variety of factors including the activity of the particular compound, the route of administration, the time of administration, the rate of excretion of the particular compound being employed, the duration of the treatment, other drugs, compounds and/or materials used in combination, the age, sex, weight, condition, general health and prior medical history of the patient being treated, and like factors well known in the medical arts. A physician or veterinarian having ordinary skill in the art can readily determine and prescribe the effective amount of the pharmaceutical composition required.

A skilled person is aware that as smaller animals have higher metabolic rates and thus smaller animals require a larger drug dose on weight basis. Dose conversions between animals, and between humans and animals, are reviewed in Nair and Jacob (J Basic Clin Pharm. March 2016-May 2016; 7(2): 27-31) and Holliday, et al., (1967 The Relation of Metabolic Rate to Body Weight and Organ Size. A Review. Pediat.Res. 1: 185-195).

In some embodiments of the methods and uses disclosed herein, at least 5 mg/day of compounds disclosed herein are provided to a human (such as by oral administration). Preferably, at least 10 mg/day of the compounds are provided. In some embodiments, a compound as disclosed herein is provided to a human at a dose of between 0.1 mg/kg to 100 mg/kg. Such amounts of the compounds are particularly useful when providing the compounds systemically (e.g., orally). A skilled person will recognize that lower amounts can be used when administered locally (e.g., on the skin, gums, wound). The compositions disclosed herein are preferably provided for at least one week or until symptoms are alleviated. While such compositions may be provided several times (e.g., one a week, once a month, twice a year, etc.), prophylactic and therapeutic effects are observed after a single use.

In some embodiments, between 1-50 g of a compound as disclosed herein is administered to a cow, e.g., for the treatment of mastitis. Preferably, at least 5 g of the compound is administered.

In some embodiments, a composition is provided comprising the compounds as disclosed herein together with one or more additional agents, such as, antibiotics (e.g., antibacterial agents, antiviral agents, anti-fungal agents), anti-inflammatory agents, anti-pyretic agents, and pain-relieving agents.

In some embodiments, the compounds disclosed herein are used together with an antimicrobial agent such as antifungal drugs or antibiotics. While not wishing to be bound by theory, the disclosure provides that the compounds disclosed herein target the biofilms. The antimicrobial drugs can then exert their effect on remaining planktonic cells as well as the microbial cells in the disrupted biofilm. As a skilled person will appreciate, the combination of an antimicrobial with the compounds described herein can reduce the dosage and/or dosage frequency of the antimicrobial. Exemplary antimicrobials which may be used in the combination treatment include antifungals such as miconazole, ketoconazole, econazole, terbinafine, ciclopirox, tolnaftate, sertaconazole, sulconazole, amphotericin b, cholorxylenol, clioquinol, butenafine, naftifine, nystatin, and clotrimazole. Exemplary antibiotics include Penicillins, Tetracyclines, Cephalosporins, Quinolones, Lincomycins, Macrolides, Sulfonamides, Glycopeptides, Aminoglycosides, and Carbapenems.

The disclosure provides compositions comprising a compound disclosed herein together with an antimicrobial. As a skilled practitioner will appreciate, the compound and an antimicrobial may also be provided separately. In some embodiments the compound and an antimicrobial therapy overlap. In some embodiments, the therapy with a compound of the invention precedes antimicrobial therapy.

In some embodiments, the compositions disclosed herein are provided as or in a food product or a functional food product. The term “functional food” as used herein, refers to those foods that are prepared not only for their nutritional characteristics, but also to fulfil a specific function, such as improving health or reducing the risk of contracting diseases. Such functional foods may also be referred to as dietary supplements or (animal) food additive. To this end, biologically active compounds, such as minerals, vitamins, fatty acids, bacteria with beneficial effects, dietary fibre and antioxidants, etc., are added thereto. Such food products may be in any form suitable for oral consumption, e.g., in the form of a liquid, gel, powder, pill, tablet, or in gel capsules.

The functional food may also include animal digest, e.g., any material that results from chemical and/or enzymatic hydrolysis of clean and undecomposed animal tissue. The functional food may also include dried brewers yeast, e.g., the dried, inactive agent that is a byproduct of the brewing industry. The animal digest and dried brewers yeast have been found to enhance the palatability of the functional food. When present in the functional food, the animal digest comprises from about 10% to about 90% of the functional food and the dried brewers yeast comprises from about 1% to about 30% of the functional food.

In some embodiments, the disclosure provides compositions comprising the therapeutic organosulfur compounds as disclosed herein together with at least one pharmaceutically acceptable carrier, diluent and/or excipient. (See e.g., Remington: The Science and Practice of Pharmacy, Alfonso R. Gennaro (Editor) Mack Publishing Company, April 1997). As used herein, the term “pharmaceutically acceptable” refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio. Furthermore, the term “pharmaceutically acceptable diluent or carrier” refers to a pharmaceutically acceptable material, composition or vehicle, such as a liquid or solid filler, diluent, excipient, solvent or encapsulating material, involved in carrying or transporting the peptide from one organ, or portion of the body, to another organ, or portion of the body.

The pharmaceutical composition may be administered by any suitable route and mode. As will be appreciated by the person skilled in the art, the route and/or mode of administration will vary depending upon the desired results. The pharmaceutical compositions may be formulated in accordance with routine procedures for administration by any routes, such as parenteral, topical (including ocular), oral, sublingual, transdermal, or by inhalation. Parenteral administration includes, e.g., intravenous, intramuscular, intra-arterial, intracoronary, intrathecal, intracapsular, intraorbital, intracardiac, intradermal, intraperitoneal, transtracheal, subcutaneous, subcuticular, intra-articular, subcapsular, subarachnoid, intraspinal, epidural and intrasternal injection and infusion. Preferred routes are oral or topical administration.

The compositions may be in any suitable forms, such as liquid, semi-solid and solid dosage forms. The compositions may be in the form of tablets, capsules, powders, granules, lozenges, creams or liquid preparations (in particular for administration to the skin or eye), such as sterile parenteral solutions or suspensions or in the form of a spray, aerosol or other conventional method for inhalation. The pharmaceutical compositions of the present invention include those suitable for oral, nasal, topical (including buccal and sublingual), rectal, vaginal and/or parenteral administration. In particular embodiments, the composition is a topical composition in the form of a cream, gel, ointment, lotion, foam, suspension, spray, aerosol, or powder aerosol. The compositions are particularly useful for administration to the skin. Suitable compositions also include oral care compositions, e.g., toothpaste, dentifrice, tooth powder, tooth gel, subgingival gel, mouthrinse/mouthwash, artificial saliva, denture product, mouthspray, lozenge, oral tablet, and chewing gum.

The disclosure also provides the in vitro use of the compositions as disclosed herein for preventing or reducing biofilm formation or growth on a surface, and/or for degradation or reduction of biofilms on a surface. Preferably, the methods are for reducing biofilm or biofilm formation on a surface. In some embodiments, the method comprises contacting a biofilm attached to a surface with the compositions disclosed herein. The compositions include, e.g., cleaning compositions. Compositions for cleaning (or rather removing or reducing) biofilms are known in the art and may include surfactants and enzymes (e.g., proteases and polysaccharidases).

Any surface may be treated with the compositions disclosed herein so as to coat such surfaces. The surfaces may be, e.g., sprayed, dipped, or soaked in the compositions. A surface includes glass, metal, porous, and non-porous surfaces. It also pertains to exterior and interior and surfaces of equipment that can be contaminated, such as those found in the food industry or the medical equipment found in hospitals and health care facilities, as well as plumbing systems (e.g., sink drain), countertops, building materials, ductwork, clean rooms. A surface also refers to the interior or exterior of pipes, for example drains, as well as swimming pools, tanks (e.g., for aquaculture), purification filters, toilet bowl, sinks, surfaces in the greenhouse. A surface also includes water, such as from a drinking trough.

In some embodiments the surface is of a medical device, such as prosthetics (hip implants, dental implants, prosthetic joint, a voice prosthetic, a penile prosthetic) a mechanical heart valve, a cardiac pacemaker, an arteriovenous shunt, a schleral buckle, catheters (e.g., central venous catheter, an intravascular catheter, an urinary catheter, a Hickman catheter, a peritoneal dialysis catheter, an endrotracheal catheter), tympanostomy tube, a tracheostomy tube, a surgical suture, a bone anchor, a bone screw, an intraocular lens, a contact lens, an intrauterine device, an aortofemoral graft, or a vascular graft. Other infections from medical devices include those from abdominal drains, biliary tract stents, breast implants, cardiac pacemakers, cerebrospinal fluid shunts, contact lenses, defibrillators, dentures, electrical dialyzers, endotracheal tubes, indwelling urinary catheters, intrauterine devices, intravenous catheters, joint prostheses, mechanical heart valves, nephrostomy tubes, orthopedic implants, peritoneal dialysis catheters, prosthetic heart valves, prosthetic joints allosplastic orthopedic devices, tissue fillers, urethral stents, vascular prostheses, ventilator-associated pneumonia, ventricular assist devices, ventricular derivations, ventricular shunts, and voice prostheses.

In some embodiments the surface is of a surgical device, such as clamp, forceps, scissor, skin hook, tubing, needle, retractor, scaler, drill, chisel, rasp, or saw.

As used herein, “to comprise” and its conjugations is used in its non-limiting sense to mean that items following the word are included, but items not specifically mentioned are not excluded. In addition the verb “to consist” may be replaced by “to consist essentially of” meaning that a compound or adjunct compound as defined herein may comprise additional component(s) than the ones specifically identified, said additional component(s) not altering the unique characteristic of the invention.

The articles “a” and “an” are used herein to refer to one or to more than one (i.e., to at least one) of the grammatical object of the article. By way of example, “an element” means one element or more than one element.

The word “approximately” or “about” when used in association with a numerical value (approximately 10, about 10) preferably means that the value may be the given value of 10 more or less 1% of the value.

The compounds and compositions disclosed herein are useful as therapy and in therapeutic treatments and may thus be useful as medicaments and used in a method of preparing a medicament. In some embodiments, the disclosure provides methods which are not a treatment of the human or animal body and/or methods that do not comprise a process for modifying the germ line genetic identity of a human being.
wherein the cell is not a human germ cell line.

All patent and literature references cited in the present specification are hereby incorporated by reference in their entirety.

The invention is further explained in the following examples. These examples do not limit the scope of the invention, but merely serve to clarify the invention.

### EXAMPLES

### Example 1: Chemical Synthesis of Thiosulfinates

General procedures for the synthesis of symmetric aliphatic thiosulfinates are well-known (see, for example, J Am Chem Soc; 1947; 69; 1710-1713). A reaction scheme is depicted below, wherein for compound 1 both R are ethyl; for compound 2 both R are propyl; and for compound 3 both R are butyl.

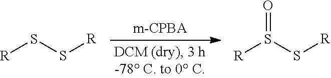

The starting materials were obtained from the following suppliers. The disulfides (diethyl disulphide, dipropyl disulphide, and dibutyl disulphide) were purchased from TCI, m-CPBA and magnesium sulphate from Sigma Aldrich, sodium hydrogen bicarbonate from Thermo Fisher, and dichloromethane from Rathburn.

The disulphide (16.36 mmol) was dissolved in dry dichloromethane (DCM, 30 mL) under a N2 atmosphere at −78° C. and meta-chloroperoxybenzoic acid (m-CPBA, 16.36 mmol), dissolved in dry DCM (30 mL), was then added dropwise. Once the addition was complete, the reaction was left to stir for 3 h, slowly warming to 0° C. The reaction was quenched with saturated sodium hydrogen bicarbonate, and the resulting aqueous solution was extracted 3 times with DCM. The combined organic fractions were then dried over anhydrous MgSO₄ and solvent was evaporated under reduced pressure. The crude product was then purified by automated flash chromatography (40 g silica, 30:70% ethyl acetate:heptane, 20 min, 28 mL/min). The product was detected by thin layer chromatography (TLC) using KMnO₄ staining. The product containing fractions were combined and the solvent was evaporated in vacuo at 40° C. to give the pure product (compounds 1-3) which is analysed by ¹H-NMR.

### Compound 1: S-ethyl ethanesulfinothioate (Diethyl thiosulfinate)

Yield: 54%, as a yellow oil; 1H-NMR 6 (400 MHz, CDCl₃): 3.25-3.05 (4H, m, O═SCH₂, SCH₂), 1.46 (3H, t, J=7.2 Hz, O═SCH₂CH₃), 1.40 (3H, t, J=7.6 Hz, SCH₂CH₃) ppm.

### Compound 2: S-propyl propane-1-sulfinothioate (Di-n-propyl thiosulfinate)

Yield: 49%, as a yellow oil; 1H-NMR 6 (400 MHz, CDCl₃): 3.18-3.00 (4H, m, O═SCH₂, SCH₂), 1.90-1.72 (4H, m, O═SCH₂CH₂, SCH₂CH₂), 1.06 (3H, t, J=7.2 Hz, (O═SCH₂CH₂CHs), 1.01 (3H, t, J=7.6 Hz, SCH₂CH₂CHs) ppm.

### Compound 3: S-butyl butane-1-sulfinothioate (Di-n-butyl thiosulfinate)

Yield: 51%, as a yellow oil; 1H-NMR 6 (400 MHz, CDCl₃): 3.10-2.90 (4H, m, O═SCH₂, SCH₂), 1.75-1.58 (4H, m, O═SCH₂CH₂, SCH₂CH₂), 1.45-1.28 (4H, m, O═SCH₂CH₂CH₂CH₃, SCH₂CH₂CH₂CH₃), 0.84 (3H, t, J=7.2 Hz, O═SCH₂CH₂CH₂CHs), 0.81 (3H, t, J=7.6 Hz, SCH₂CH₂CH₂CHs) ppm.

### Example 1b

Experimental design: Oxidation of a disulfide leads to the formation of the corresponding thiosulfinate and thiosulfonate. For the oxidation reaction, commercially available disulfides were used and mcpba (meta-chloroperoxybenzoic acid) was used as an oxidant in organic synthesis. This leads to the formation of the corresponding thiosulfinates.

General Procedure for the synthesis of symmetric thiosulfinates (J Am Chem Soc; 1947; 69; 1710-1713). The disulfide (16.36 mmol) was dissolved in dry DCM (=dichloromethane 30 mL) under a N2 atmosphere at −78° C. and m-CPBA (meta-chloroperoxybenzoic acid 16.36 mmol), dissolved in dry DCM (30 mL), was dropwise added. Once the addition was complete, the reaction was left to stir for 3 h, slowly warming up to 0° C. The reaction was quenched with saturated sodium hydrogen bicarbonate, and the resulting aqueous solution was extracted 3 times with DCM. The combined organic fractions were subsequently dried over anhydrous MgSO₄ and solvent was evaporated under reduced pressure. The crude product was then purified by automated flash (Supplier: Teledyne Isco). The product was detected by thin layer chromatography (TLC) using KMnO₄ staining. The product containing fractions were combined and the solvent was evaporated in vacuo at 40° C. to give the pure product which is analysed by 1H-NMR. Remark: in particular attention is paid when evaporating the solvent for the aliphatic, F-aryl and the heterocyclic analogues. The temperature is kept low (around 25° C.) to prevent degradation or evaporation due to volatility. General procedure for the synthesis of symmetric thiosulfonates (Chem. Eur. J. 2015, 21, 8105). At 30° C. to acetic acid (2.34 ml) the disulfide (0.012 mol) was added followed by hydrogen peroxide (30% Wt, 10.89 ml) over approximately 5 min. The temperature was increased to 55° C. and the reaction was stirred at this temperature for approximately 4 hr. The reaction was cooled to room temperature and treated with 5M NaOH till the pH was basic. Hereafter, the raw material was extracted 3 times with DCM. The combined organic extracts were dried over anhydrous MgSO₄ and the solvent was evaporated under reduced pressure to obtain the crude mixture. Hereafter, purification takes place by automated flash (Teledyne ISCO) (table 1 and 2). The product was detected by TLC using KMnO₄ staining and the product containing fractions were combined and the solvent was evaporated in vacuo to give the pure product which was analysed by 1H-NMR. Remark: for aliphatic and heterocyclic after addition of the oxidant the reaction is set to 40° C. S-propyl-4-methylbenzene thiosulfonate was prepared according to Angew. Chem. Int. Ed., 2018, 57, 12290-12293.

Results. When hydrogen peroxide was used as the oxidant in the presence of acetic acid the formation of corresponding thiosulfonates was formed. Most compounds were synthesized in standard reaction conditions with good to moderate yield as outlined (in Table 1). Some, in particular the aliphatic and heterocylic, required benign reaction conditions in thiosulfonation compared to the aryl substituted to get some reaction product. The benzyl substituted is more reactive in comparison with other aryl substituted analogues and that might be the reason for low reaction product in tried reaction conditions. The heterocyclic analogues (dibenzothiazolyl disulfide) did not undergo a reaction due to solubility issue and resulted in tar with unsoluble solid. The reaction for its thiosulfinate was attempted in 2 solvents (dichloromethane and chloroform) with the same outcome. The reaction for the corresponding thiosulfonate was attempted with increased temperature also without success. The oxidation of furfuryl disulfide was also troublesome due to instability. It resulted in low amount of thiosulfinate while the reaction of the corresponding thiosulfonate resulted in tar. The synthesized F-aryl thiosulfinate/thiosulfonate were initially purified using reverse phase chromatography and analysed by 1H NMR (table 1 and 2).

The compounds were not stabile for high temperatures and degraded during working up. Therefore they were resynthesized and purified by silica chromatography.

TABLE 1
Yield, purification and detection by 1H NMR during synthesis of symmetrical thiosulfinates.

| | | Purification by | | |
|---|---|---|---|---|
| | | flash | | Yield (%)/ |
| Substituent | Corresponding thiosulfinate | chromatography | Analysis by 1H NMR (400 MHz, CDCl3) | appearance |
| Propyl | Di-n-propyl thiosulfinate | Si 40 gr, | δ 3.18-3.00 (m, 4H), 1.90-1.72 (m, 4H), 1.06 (t, 3H, J = 7.2 | 40/colorless oil |
| | | EtOAc:Petr.ether | Hz), 1.01 (t, 3H, J = 7.6 Hz) ppm | |
| | | * | | |
| Ethyl | Di-ethyl thiosulfinate | Si 12 gr, | δ 3.25-3.05 (m, 4H), 1.46 (t, 3H, J = 7.2 Hz), 1.40 (t, 3H, | 54/yellow oil |
| | | EtOAc:Petr.ether | J = 7.6 Hz) ppm | |
| Butyl | Di-n-butyl thiosulfinate | Si 40 gr, | δ 3.10-2.90 (m, 4H), 1.75-1.58 (m, 4H), 1.45-1.28 (m, 4H), | 49/colorless oil |
| | | EtOAc:Petr.ether | 0.84 (t, 3H, J = 7.2 Hz), 0.81 (t, 3H, J = 7.6 Hz) ppm | |
| Isopropyl | S-isopropyl propane-1 thiosulfinate | Si 12 gr, | δ 3.60 (hept, 1H, J = 6.8 Hz), 3.18 (hept, 1H, J = 6.8 Hz), | 45/pale yellow oil |
| | | EtOAc:Petr.ether | 1.42-1.48 (m, 6H), 1.33-1.37 (m, 6H) ppm | |
| Phenyl | Di-phenyl thiosulfinate | C18 15.5 gr, | δ 7.66 (dd, J = 6.8, 2.8 Hz, 2H), 7.56-7.36 (m, 8 H) ppm | 36/yellow solid |
| | | ACN:H2O | | |
| F-aryl | Bis(4-fluorophenyl) thiosulfinate | Si 40 gr, | δ 7.62-7.58 (m, 2H), 7.46-7.42 (m, 2H), 7.18 (t, J = 8.4 Hz, | 25/yellow solid |
| | | EtOAc:Petr.ether | 2H), 7.08 (t, J = 8.4 Hz, 2H) ppm | |
| Cl-Aryl | Bis(4-chlorophenyl) thiosulfinate | C18 15.5 gr, | δ 7.58-7.54 (m, 2H), 7.49-7.46 (m, 2H), 7.43-7.35 (m, 4H) | 22/white solid |
| | | ACN:H2O | ppm | |
| CH3O-Aryl | Bis(4-methoxiphenyl) thiosulfinate | C18 15.5 gr, | δ 7.58 (d, J = 8.4 Hz, 2H), 7.44 (d, J = 8.4 Hz, 2H), 7.00 | 37/pale yellow |
| | | ACN:H2O | (d, J = 8.4 Hz, 2H), 6.91 (d, J = 8.4 Hz, 2H), 3.88 (s, 3H), | solid |
| | | | 3.85 (s, 3H) ppm | |
| Tolyl | Bis(p-tolyl) thiosulfinate | C18 15.5 gr, | δ 7.59 (d, J = 8.0 Hz, 2H), 7.46 (d, J = 8.4 Hz, 2H), 7.32 | 14/pale yellow |
| | | ACN:H2O | (d, J = 8.0 Hz, 2H), 7.21 (d, J = 8.0 Hz, 2H), 2.44 (s, 3H), | solid |
| | | | 2.41 (s, 3H) ppm | |
| Benzyl | Di-benzyl thiosulfinate | C18 15.5 gr, | δ 7.40-7.34 (m, 10H), 4.35-4.30 (m, 4H) ppm | 17/light pink solid |
| | | ACN:H2O | | |
| Furfuryl | Di-furfuryl thiosulfinate | Si 40 gr, | δ 7.42 (dd, J = 0.8, 2.0 Hz, 2H), 6.36 (dd, J = 1.6, 3.2 Hz, | 56/brown oil |
| | | EtOAc:Petr.ether | 2H), 6.26 (d, J = 3.2 Hz, 2H), 3.72 (s, 4H) ppm | |

n.a.: not applicable because of commercial availability
* means: 40 gram silica column; EtOAc:Petr.ether is liquid phase

TABLE 2
Yield for the synthesis of symmetrical thiosulfonates

| | | Purification by flash | | Yield (%)/ |
|---|---|---|---|---|
| Substituent | Corresponding thiosulfonate | chromatography | Analysis by 1H NMR (400 MHz, CDCl3) | appearance |
| Butyl | Di-n-butyl thiosulfonate | C18 15.5 gr, | δ 3.30 t, J = 8.0 Hz, 2H), 3.13(t, J = 7.6 Hz, 2H), 1.91-1.87 (m, | 20/colorless oil |
| | | ACN:H2O | 2H), 1.76-1.67 (m, 2H), 1.51-1.41 (m ,2H), 0.97, J = 7.6 Hz, | |
| | | | 3H), 0.93 (t, J = 7.6 Hz, 3H) ppm | |
| Isopropyl | D-isopropyl thiosulfonate | Si 12 gr, | δ 3.65-3.62 (m, 1H), 3.23-3.2 (m, 1H), 1.50-1.47 (m, 6H), | 6/pale yellow |
| | | EtOAc:Petr.ether | 1.41-1.38 (m, 6H) ppm | oil |
| F-aryl | Bis(4-fluorophenyl) | C18 15.5 gr, | δ 7.63-7.60 (m, 2H), 7.41-7.37 (m, 2H), 7.17-7.06 (m, 4H) | 26/off-white |
| | thiosulfonate | ACN:H2O | ppm | solid |
| Cl-Aryl | Bis(4-chlorophenyl) | C18 15.5 gr, | δ 7.54-7.52 (m, 2H), 7.44-7.42 (m, 2H), 7.37-7.31 (m, 4H) | 5/off white |
| | thiosulfonate | ACN:H2O | ppm | solid |
| CH3O-Aryl | Bis(4-methoxyphenyl) | Si 40 gr, | δ 7.51 (d, J = 8.8 Hz, 2H), 7.28 (d, J = 8.8 Hz, 2H), 6.88 | 18/pale yellow |
| | thiosulfonate | EtOAC:petr.ether | (d, J = 8.8 Hz, 2H), 6.86 (d, J = 8.8 Hz, 2H) ppm | solid |
| Benzyl | Di-benzyl thiosulfonate | C18 15.5 gr, | δ 7.44-7.39 (m, 5H), 7.31-7.31 (m, 5H), 4.25 (s, 2H), 4.07 (s, | 9/White solid |
| | | ACN:H2O | 2H) ppm | |

n.a.: not applicable because of commercial availability
* means: 40 gram silica column; EtOAc:Petr.ether is liquid phase

### Example 2: MIC and Biofilm Assays

The MIC assay in this example measures growth inhibition of planktonic microorganisms. As shown herein, a number of compounds tested show an effect on planktonic bacteria.

As will be appreciated by a skilled person, the effect of a compound against planktonic microorganisms is not indicative of the effect of such compounds against a biofilm.

This has been demonstrated for a number of antimicrobials as previously discussed herein and is also well-known in the literature. See, e.g., Roy et al. which indicates that bacteria in biofilms have increased resistance against conventional antibiotics by around 1000-fold (2018 Virulence 9:522-554).

In contrast to studies concerned with planktonic microorganisms, the Biofilm eradication assay in this example demonstrates the effect of compounds against a biofilm.

The main mechanisms of action of a compound in a biofilm assay are believed to be related to either the inhibition of quorum sensing and the following collective gene switch resulting in adhesion of bacteria and the synthesis or to the assembly of extracellular matrix to form the 3-dimensional structure of a biofilm. These mechanisms are entirely different from a pure antibacterial/antimicrobial “killing” effect.

If a compound tested effectively in a biofilm assay, but has no antibacterial effect, then the compound may be combined with an antibiotic to ensure that the bacteria released from the biofilm are killed. This is especially the case if the immune system of the animal is compromised. However, such compounds are also particularly useful at specifically targeting biofilms, while have little or no effect on, e.g., healthy gut flora. Such compounds are particularly preferred.

TABLE 3
Compounds with CAS number and supplier used in this study.

| Compound | CAS number | Supplier |
|---|---|---|
| Di-methyl disulfide | 624-92-0 | Sigma Aldrich |
| Di-ethyl disulfide | 110-81-6 | Sigma Aldrich |
| Di-n-propyl disulfide | 629-19-6 | Sigma Aldrich |
| Di-n-butyl disulfide | 629-45-8 | Merck |
| Di-phenyl disulfide | 882-33-7 | Sigma Aldrich |
| Di-isopropyl disulfide | 4253-89-8 | Acros |
| Di-benzyl disulfide | 150-60-7 | TCI |
| Bis (4-fluorophenyl) disulfide. | 405-31-2 | Fischer Scientific |
| Methyl propyl disulfide | 2179-60-4 | TCI |
| Bis(4-methoxyphenyl) disulfide | 5335-87-5 | Thermo Scientific |
| Di-p-tolyl disulfide | 103-19-5 | Sigma Aldrich |
| Di-furfuryl disulfide | 4437-20-1 | TCI |
| Bis(4-chlorophenyl) disulfide | 1142-19-4 | Thermo Scientific |
| Di-n-hexyl disulfide | 10496-15-8 | commercially available |
| Di-n-decyl disulfide | 10496-18-1 | commercially available |
| Di-(n-Propyl) thiosulfinate | 1948-52-3 | Organic synthesis |
| Bis (p-tolyl) thiosulfinate | 6481-73-8 | Organic synthesis |
| Di phenyl thiosulfinate | 1208-20-4 | Organic synthesis |
| Di-isopropyl thiosulfinate | 38739-06-9 | Organic synthesis |
| Di-benzyl thiosulfinate | 16302-98-0 | Organic synthesis |
| Bis(4-chlorophenyl) thiosulfinate | 6481-72-7 | Organic synthesis |
| Bis(4-fluorophenyl) thiosulfinate | 61169-14-0 | Organic synthesis |
| S-decyl-1-decane thiosulfinate | 56527-90-3 | Organic synthesis |
| Di (n-Propyl) thiosulfonate | 1113-13-9 | Syncom |
| Di-methyl thiosulfonate | 2949-92-0 | Sigma Aldrich |
| Di-phenyl thiosulfonate | 1212-08-4 | Sigma Aldrich |
| Di-isopropyl thiosulfonate | 10027-69-7 | Organic synthesis |
| Di-benzyl thiosulfonate | 16601-40-4 | Organic synthesis |
| Bis(4-Chlorophenyl) thiosulfonate | 1146-44-7 | Organic synthesis |
| Bis(4-Fluorophenyl) thiosulfonate | 2905-15-9 | Organic synthesis |
| S-propyl-4-methylbenzene | 90494-81-8 | Organic synthesis |
| thiosulfonate | | |

### 1.1 Dissolving and Dilution of Compounds

In an Eppendorf tube between 10-20 mg of a compound as indicated in table 3 was weighed. The exact weighed amount was noted and the volume of solvent to reach a 80 mM dilution calculated. Half of the volume was added first, by adding Tween 80. In a second step, the other half of the volume DMSO was added and the closed tube inverted 5-10 times and then vortexed very thoroughly for at least 30 seconds. If after this procedure unsolved material was still visible, the tube was placed in a shaker at 37° C. at 150 rpm for at least 30 minutes. The dissolved and transparent dilutions had a concentration of 80 mM and were diluted 10 times with Mueller Hinton Broth 2 cation adjusted (MHB-II) for the MIC assay (method 1.2). For the MBEC assay (method 1.3) the solution needs to be diluted to a final concentration of 2 mM in 0.9% saline solution and then further diluted to 1 mM, 0.5 mM, 0.25 mM and 0.125 mM in saline. Those dilutions were added as treatment to the 96 well plates with the grown biofilm (see method 3).

### 1.2 MIC Assays

Frozen aliquots of Staphylococcus epidermis ATCC 35984 and Pseudomonas aeruginosa ATCC 27853 were used to inoculate a tryptic soy agar (TSA) plate and incubated overnight at 37° C. After incubation 3-5 well-isolated colonies with the same morphology were selected from the TSA plate and resuspended in 2 mL 0.9% saline solution with 3-6 glass beads and vortexed. The optical density at 600 nm (OD₆₀₀) of the bacterial suspension was measured with a spectrophotometer (Evolution™ 201/220 UV-Visible Spectrophotometer, Thermo Fisher Scientific) and diluted to an OD₆₀₀ of 0.0008, which resembles 106 CFU/mL.

The MIC assay was performed in a flat-bottom 96-microtiter plate. 50 μL of MHB-II was added into columns 2-11, and 100 μL to column 12 for sterility control. 100 μL of the 8 mM test compound solution (method 1) was added to column 1. A two-fold dilution series of the solution in MHB-II was achieved by resuspending 50 μL of the 8 mM solution from column 1 to column 2. This step was repeated until column 10. The respective concentration of Tween 80 and DMSO in column 1 was 2.5%, which is two-fold diluted throughout the plate. 50 μL of the bacterial suspension was added to column 1-11. The microtiter plates were sealed with adhesive polyethylene film for sealing microplates (Diversified Biotech) and incubated for 24 h at 37° C. After incubation the OD was measured at 600 nm with the Varioskan (Thermo Fisher Scientific). The minimal concentration which causes reduction of final OD₆₀₀ of the bacterial culture by 50% (MIC50) and by 90% (MIC90) was calculated after normalization of the obtained OD values to the growth control in column 11. This assay was repeated three times in three independent experiments.

### 1.3 MBEC Assays

Frozen aliquots of Staphylococcus epidermis ATCC 35984 and Pseudomonas aeruginosa ATCC 27853 were used to inoculate 20 mL of Tryptic Soy Broth (TSB) in a 100 mL Erlenmeyer flask at 37° C. and 150 rpm and incubated overnight (Infors). The OD₆₀₀ of the overnight culture was measured with a spectrophotometer (Evolution™ 201/220 UV-Visible Spectrophotometer, Thermo Fisher Scientific) and diluted to an OD₆₀₀ of 0.2, which resembles 10⁸ CFU/mL. The cell suspension of OD₆₀₀=0.2 was seeded into a U-bottom-shaped 96 well plates by pipetting 100 μL per well in column 1-11, column 12 was filled with 100 μL medium as sterility control. The 96 well plates were sealed with adhesive polyethylene film for sealing microplates and incubated for 48 h at 37° C. at static conditions. After the biofilm growth phase, 100 μL of treatment solutions (method 1) were added to the vials resulting in treatment concentrations of 1 mM, 0.5 mM, 0.25 mM and 0.125 mM and with the respective concentration of Tween 80 and DMSO each at 0.63%, 0.32%, 0.16% and 0.08%. The same concentration was applied to four different wells of the same bacterial strain, resulting in four replicates for each concentration and strain. The treatment was incubated for 20 h at 37° C. at static conditions. After the treatment the supernatant was carefully removed and the well with the biofilm washed carefully for one time with 200 μL 0.9% saline solution. 200 μL saline solution was added and the biofilm was resuspended thoroughly. When fully homogenized then 20 μL was used for the first dilution in 180 μL 0.9% saline solution, which was prepared in a second plate. This dilution was continued until dilution step 10E−5 and 100 μL of the 10E−4 and 10E−5 dilution plated on a TSA plate. The TSA plates were incubated at 37° C. for 24 h. The colonies on the plates were enumerated, the colony forming units per mL of the dispensed biofilm and averages thereof and the log reduction in comparison with the untreated sample from the same wells plate calculated. Reduction of colony forming units goes hand in hand with the thickness of the biofilm and thus with the number bacteria that are present in the biofilm. Therefore, a significant reduction of colony forming units in the biofilm was defined at the value of log reduction ≥1 or more. This assay was repeated two times in two independent experiments)

### 1.4 MBIC Assays

Frozen aliquots of Staphylococcus epidermis ATCC 35984 and Pseudomonas aeruginosa ATCC 27853 were used to inoculate a tryptic soy agar (TSA) plate and were incubated overnight at 37° C. After incubation 3-5 well-isolated colonies with the same morphology were selected from the TSA plate and resuspended in 2 mL 0.9% saline solution with 3-6 glass beads and vortexed. The optical density of the bacterial suspension was measured at 600 nm (OD₆₀₀) with a spectrophotometer (Evolution™ 201/220 UV-Visible Spectrophotometer, Thermo Fisher Scientific) and diluted to an OD₆₀₀ of 0.2. The MBIC assay was performed in a 96-microtiter plate. 50 μL of TSB was added into columns 2-11, and 100 μL to column 12 for sterility control. 100 μL of the 8 mM test compound solution (method 1) was added to column 1. A two-fold dilution series of the solution in TSB was achieved by resuspending 50 μL of the 8 mM solution from column 1 in column 2. This step was repeated until column 10. The respective concentration of Tween 80 and DMSO in column 1 is at 2.5%, which is two-fold diluted throughout the plate. 50 μL of the bacterial suspension was added to column 1-11. The microtiter plates were sealed with adhesive polyethylene film for sealing microplates (Diversified Biotech) and incubated for 48 h at 37° C. After incubation the supernatant is carefully removed and the well with the biofilm washed for one time with 100 μL 0.9% saline solution. 100 μL 0.1 M HCl was added and incubated for 1 h at room temperature to fix the biofilm. After incubation the HCl was removed and 100 μL crystal violet (0.1% v/v in water) was added and incubated for 30 min at room temperature. The unbound crystal violet was removed and the wells washed one time with 100 μL demineralized water. 100 μL 30% acetic acid was added and incubated for 1 h at 37° C. and 150 rpm. The solution was resuspended and transferred to a flat-bottom 96-well plate and the absorbance measured at 540 nm with the spectrophotometer. This assay was repeated two times in two independent experiments.

Results and discussion. In table 4 the results of the tests are presented.

When a biofilm is eradicated or degraded, the microorganisms are released into the environment. In case where the biofilm is present in vivo, e.g., in a human or animal, the released microorganisms can then be attacked by the immune system. The advantage of the present in vitro system is that it gives the opportunity to investigate the effects of agents on a biofilm and the respective microorganisms as such, without additional effects of the environment (e.g., the immune system).

In table 4, a number of organosulfur compounds show low MIC-values, whereas the MBEC values were high. Table 4 also depict a number of compounds that exhibit high MIC-values and very low MBEC-values. This is a very surprising result. This suggests that the growth of S. epidermis and P. aeruginosa is not affected by these organosulfur compounds, but rather that the compounds have a strong effect against biofilms.

TABLE 4
Results of the MIC- and biofilm assays. MIC50 en MIC90 were defined as the minimal concentration which causes reduction of
final OD600 of the bacterial culture by 50% (MIC50) and by 90% (MIC90) compared to a culture without the test compound.

| | | | | | | S. | P. | | |
|---|---|---|---|---|---|---|---|---|---|
| | | | | | | epidermidis | aeruginosa | | |
| | | | | | | MBEC | MBEC | S. | P. |
| | | S. epidermidis | | P. aeruginosa | | mM with | mM with | epidermidis | aeruginosa |
| | | MIC50 | MIC90 | MIC50 | MIC90 | log | log | MBIC90 | MBIC90 |
| Class | Compound | [mM] | [mM] | [mM] | [mM] | reduction ≥1 | reduction ≥1 | [mM] | [mM] |
| di-alkyl disulfides | Di-methyl disulfide | >4 | >4 | >4 | >4 | >1 | >1 | | |
| | Di-ethyl disulfide | >4 | >4 | >4 | >4 | >1 | >1 | | |
| | Di-n-propyl disulfide | >4 | >4 | >4 | >4 | >4 | >4 | 4 | >4 |
| | Di-n-butyl disulfide | >4 | >4 | >4 | >4 | >1 | >1 | | |
| | Di-phenyl disulfide | 0.125 | >4 | 2 | >4 | >1 | 0.125 | | |
| | Di-isopropyl disulfide | >4 | >4 | >4 | >4 | >1 | >1 | | |
| | Di-benzyl disulfide | >4 | >4 | >4 | >4 | >1 | >1 | | |
| | Methyl propyl disulfide | >4 | >4 | >4 | >4 | >1 | >1 | | |
| | Bis (4-methoxyphenyl) disulfide | >4 | >4 | >4 | >4 | >1 | >1 | | |
| | Di-p-tolyl disulfide | >4 | >4 | >4 | >4 | >1 | >1 | >4 | 4 |
| | Di-furfuryl disulfide | 2 | >4 | 2 | >4 | >1 | >1 | | |
| | Di-n-hexyl disulfide | >4 | >4 | >4 | >4 | >1 | >1 | | |
| | Di-n-decyl disulfide | >4 | >4 | >4 | >4 | >1 | >1 | | |
| halogenated di- | Bis (4-chlorophenyl) disulfide | 0.01 | 0.03 | 0.02 | 0.03 | >1 | >1 | >4 | >4 |
| alkyl disulfides | Bis (4-fluorophenyl) disulfide | 0.06 | 0.06 | 0.125 | 0.25 | >1 | >1 | | |
| | Di-n-propyl thiosulfinate | 0.25 | 0.5 | 1 | 2 | 2 | 4 | >4 | 2 |
| di-alkyl | Bis (p-tolyl) thiosulfinate | 0.125 | 0.25 | 0.125 | 0.25 | 1 | 1 | >4 | >4 |
| thiosulfinates | Di-phenyl thiosulfinate | 1 | 1 | 1 | 1 | 2 | 2 | >4 | >4 |
| | Di-isopropyl thiosulfinate | 2 | 2 | 2 | 4 | 0.5 | 0.125 | >4 | >4 |
| | D-benzyl thiosulfinate | 0.03 | 0.03 | 0.06 | 0.5 | 1 | 0.125 | 4 | >4 |
| | S-decyl-1-decane thiosulfinate | >4 | >4 | >4 | >4 | >1 | >1 | | |
| halogenated di- | Bis (4-chlorophenyl) thiosulfinate | 0.02 | 0.02 | 0.02 | 0.02 | >1 | >1 | | |
| alkyl | Bis (4-fluorophenyl) thiosulfinate | 0.125 | 0.25 | 1 | 4 | >1 | >1 | | |
| thiosulfinates | | | | | | | | | |
| di-alkyl | Di-n-propyl thiosulfonate | 0.25 | 0.25 | 0.25 | 0.25 | 0.25 | 1 | 2 | 0.5 |
| thiosulfonate | Di-methyl thiosulfonate | 0.25 | 0.25 | 0.25 | 0.5 | 2 | 2 | >4 | 4 |
| | Di-phenyl thiosulfonate | 1 | 2 | 2 | 2 | 2 | >4 | | |
| | Di-isopropyl thiosulfonate | 2 | 2 | 2 | 2 | >1 | >1 | | |
| | Di-benzyl thiosulfonate | 2 | 4 | 4 | >4 | 0.125 | 0.125 | | |
| | S-propyl-4-methylbenzene | 0.06 | 0.13 | 0.06 | 0.06 | 0.5 | >1 | | |
| | thiosulfonate | | | | | | | | |
| | | | | | 4 | | | | |
| Halogenated di- | Bis (4-chlorophenyl) thiosulfonate | 0.03 | 0.06 | 0.25 | 2 | >1 | >1 | | |
| alkyl | Bis (4-fluorophenyl) thiosulfonate | 0.25 | 2 | 2 | 2 | >1 | >1 | | |
| thiosulfonates | | | | | | | | | |

### Example 3 In Vivo Tests—Mastitis

To demonstrate the in vivo effects of the compounds disclosed herein, a number of organosulfur containing compositions were tested on a number of biofilm-related disorders in vivo. Bovine mastitis is a disease affecting worldwide millions of cows annually. The disease is caused by a variety of very different bacteria and in some cases also yeasts that invade the mammary gland of lactating cows, causing a persistent infection and an inflammatory response. Bovine mastitis can be caused various Gram-positive as well as Gram-negative pathogens and the prevalence of individual pathogens may vary to some extent between countries and continents. The most prominent clinical sign of all forms of mastitis is an undesirable increase in the number of somatic cells in the milk intended for human consumption caused by a (chronic) inflammatory response of the udder tissue. This secondary inflammation decreases milk production and hence causes serious economic losses for the farmer. Over the last decades, numerous pharmaceutical products have developed containing different classes of antibiotics, given alone or in combination to treat the primary infectious agents of mastitis. These pharmaceutical products are given systemically (by injection) or locally via the teat channel to fight the bacterial infections. However, despite these great efforts, bovine mastitis remains the most prevalent disease in dairy cow, as antibiotic therapy in generally only temporarily effective and, in many causes, somatic cell counts (SCC), as a clinical marker of mastitis remained high.

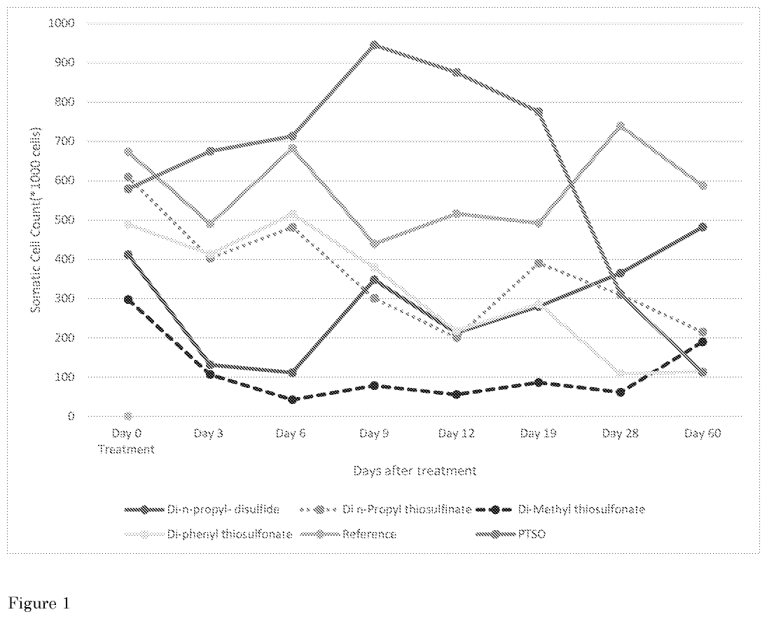

Mastitis is a known biofilm-related disorder. The formation of bacterial biofilms has been demonstrated in in vitro experiments cultivating mastitis pathogen under conditions favoring bacterial biofilm formation (quantifiable after staining and by measuring the genes that drive biofilm formation) as well as in situ by staining the biofilm matrix in the infected bovine udder tissue. For example, FIG. 1 of Schönborn S and Krömker V (2016 Journal Veterinary Microbiology, 30; 196:126-128) which depicts a biofilm matrix from udder tissue from cows suffering from mastitis.

Biomedical research of the last decennia revealed that biofilm formation is not only occurring in cases of udder infection and bovine mastitis but is a general trait of microorganisms invading human and animal tissues and causing persistent infections and inflammation. To date, microbial biofilms are one of the major unresolved challenges in modern therapy of infectious diseases in humans and animals. Clinical evidence provided in the examples below revealed an unexpected therapeutic effect of a number of organosulfur containing compositions in the treatment of important biofilm diseases such as bovine mastitis and infected chronic wounds (e.g., UCD). Several field trails are described which demonstrate that the application of the compounds is directly related to a long-lasting stable decrease of somatic cell counts in treated cows, suggesting bacterial cure and tissue regeneration. Further examples describe the effects on biofilm-related chronic wounds (i.e. Udder Cleft Dermatitis (UCD). These findings are remarkable, as it is generally recognized that common antibiotics fail to be effective against biofilm infections. These observations regarding antibiotics have been described in numerous scientific articles from human and veterinary medicine, all suggesting the formation of biofilms as the major cause of recurrent and persistent infections and tissue inflammation and damage. The presented experimental (in vitro) and in vivo (clinical observations) efficacy of the compounds described herein against prototypical biofilm infections demonstrates that these compounds can effectively be used in clinical practice to prevent and resolve biofilm infections. Biofilm formation and resolution occurs as a direct interaction between the molecules and microbes and is therefore independent of the host (animal or human). As the mechanism involved in biofilm formation are highly conserved between bacterial species, it can be assumed also that the compounds are effective against a broad spectrum of biofilm infections in humans and animals. The examples also demonstrate that the compounds can be administered orally and exert effects at distinct sites (e.g., mammary glands and UCD).

### Scope

Experiments were performed to examine the effect of compounds on the cell count or somatic cell count (SCC) in milk. The tested compounds and total dosages administered are indicated in table 5.

Somatic cell counts (SCC) is related to the amount of pathogens that are in the quarter visible for the immune system. The cell count of each cow was measured with the milk production registration (MPR). This MPR is done periodically every 30-40 days at all farms. The Somatic Cell Count (SCC) is a main indicator of milk quality. The majority of somatic cells are leukocytes (white blood cells)—which become present in increasing numbers in milk usually as an immune response to a mastitis-causing pathogen—and a small number of epithelial cells, which are milk-producing cells shed from inside of the udder when an infection occurs.

Inflammation often takes place in one of the udder quarters, resulting in the cell count to rise in this udder. However due to the dilution with the other quarters of the udders, the total increase in cell count will be lower. The SCC is quantified as the number of cells per ml of milk. SCC gives an indication of the presence of an (subclinical) udder infection with pathogens causing for instance mastitis and a main indicator of milk quality. The relationship of SCC and mastitis is reviewed in Sharma et al., 2011 Asian-Aust J Anim Sci 24:429-438.

The SCC data was extracted from milk production registrations (MPRs). MPRs are databases showing all details (e.g. SCC) of the produced milk per individual cow. MPRs of the selected farms were available periodically. SCC is quantified as the number of cells per ml of milk. In general terms: an individual cow with a value for SCC of 100,000 or less indicates an ‘uninfected’ cow and there were no significant production losses due to subclinical mastitis. A threshold SCC above 250,000 indicates infection and counts between 100,000 to 250,000 indicate a high risk of infection.

TABLE 5
Compounds and corresponding total amounts
that were orally administered to cows.

| | Cas | Total amount | Number of |
|---|---|---|---|
| Test Compound | number | in tablet (g) | cows tested |
| Control | n.a. | 0 | 1 |
| PTSO | 1113-13-9 | 4.5 | 5 |
| Di-n-propyl disulfide | 629-19-6 | 10 | 3 |
| Di-n-propyl thiosulfinate | 1948-52-3 | 10 | 1 |
| Di-methyl thiosulfonate | 2949-92-0 | 10 | 1 |
| Di-phenyl thiosulfonate | 1212-06-4 | 10 | 1 |

n.a.: not applicable

### Study Design

The experiments were performed on a farm with 90 cows. Examination was performed on cell count with the above described tablets during 60 days.

Cows with a SCC of more as 250.000 cells/ml before treatment were selected for administration of a single tablet.

The counts and average somatic cell counts of the milk of the cows are shown in FIG. 1. The SCC of the reference cows remained relatively high (see FIG. 1; compare around 673 on day 0 to 587 at day 60). In contrast, the SCC was significantly decreased in cows receiving a single treatment of di-n-propyl thiosulfinate, di-methyl thiosulfonate, or di-phenyl thiosulfonate. As a clinically significant effect was observed in the udder tissue, this suggest that the compounds not only pass the rumen (indicating stability to rumen microorganisms), but also reach the post hepatic blood stream (indicating minor or insignificant hepatic biotransformation and inactivation), and ultimately the mammary gland in a concentration that is therapeutically effective.

### Example 4: In Vivo Tests—Udder Cleft Dermatitis

A category of chronical infections are wounds that are infected by biofilm-forming microorganisms. Once the wound is infected, the microorganisms start to form a biofilm that remains attached to the wounds. The production of microbial EPS (Extracellular Polymeric Substances) helps the biofilm to form a complex, three-dimensional structure within a few hours. These complex structures are resistant to defence mechanisms in the wound. When antibiotics are applied to attack bacteria, they may only partially eradicate the biofilm and the wound and underlying tissues remain infected. These biofilms are known to lead to chronic infections and non-healing wounds. In the United States, around 16 million new biofilm-based infections are diagnosed every year. Hence biofilms constitute a major obstacle to wound healing. Examples of such wounds infecting pathogenic microorganisms are bacteria (Gram positive bacteria, for example Staphylococcus aureus; Streptococci; gram negative bacteria, for example Treponema spp., Escherichia coli, Yersiania pestis, Pseudomonas aeruginosa; yeast/fungi, for example Candida spp (albicans), Cladosporidium herbarum, Trichosporum, Rhodosporidium, Malassezia.

An example of digital dermatitis is Digital dermatitis of claws (synonyms are hairy heel warts, strawberry foot rot, mortellaro disease, Italian foot rot, papillomatous digital dermatitis) and is an infection that causes lameness with cattle.

An example of a biofilm-related disorder is Udder Cleft Dermatitis (UCD), which relates to a chronic wound. In this Example, di-alkyl thiosulfonates, dialkyl thiosulfinates and di-alkyl disulfides were tested for their effect on cows affected by UCD.

UCD is a skin lesion located at the anterior junction between the udder and the abdominal wall or between the front quarters of the udder. The lesions may vary in appearance and size, but thickened skin, crusts, pus, and wounds that easily bleed are common findings. Udder cleft dermatitis can be difficult to detect due to its anatomical position and the fact that affected cows seldom show general signs of disease. Few studies on UCD prevalence have been published, and most have included only one or a few herds, mainly categorized as problem herds. The within-herd prevalence in those studies varied between 0 and 22%. In a recent Dutch study, however, 20 herds were included, of which 3 had no UCD, whereas the within-herd prevalence in the other herds varied between 2.5 and 13% (Amersfort et al., 2012). The etiology of UCD is unclear, but several factors, such as udder conformation and udder edema have been suggested to play a role. Cow factors such as parity and DIM (days in milk) have also been associated with UCD (Beattie and Taylor, 2000, J. Brit. Cattle Vet. Assoc. 8, 377-380).

Lesions are most commonly identified on the plantar aspect of the interdigital cleft of the hind limbs. Treponema spp are routinely present in large numbers of active lesions. Lesions are painful to the touch and can result in clinical lameness. The infectious nature generally results in endemic infection of cattle herds and is responsible for large economical losses.

### Study Design

Experiments were performed to examine the effect of various sprays containing water (control), a test compound, or a reference compound (CTC spray, which contains chlorotetracycline hydrochloride, and was obtained from Dechra veterinary products).

0.35 mL of the spray was sprayed on an udder cleft wound per treatment. Treatments took place once a day at t=0, at t=3 days and t=6 days. The result of these experiments are indicated in Table 7. In Table 6, the assessment qualifications that are used in Table 7, are explained.

TABLE 6
Assessment qualification and corresponding description of the
udder cleft wound. A score of 1 indicates the worst wound, and
a score of 10 indicates that wound had completely healed.

| Assessment | |
|---|---|
| qualification | Description |
| 1 | Chronic and slimy wounds with pus, thick wound crusts, |
| | clearly inflamed, white and sensitive wounds with proud |
| | flesh |
| 2 | Chronic and slimy wounds with pus, thick wound crusts, |
| | clearly inflamed, white and sensitive |
| 3 | Chronic and slimy wounds with pus, thick wound crusts, |
| | clearly inflamed, red and sensitive |
| 4 | slimy wounds, thick wound crusts, clearly inflamed, |
| | red and sensitive |
| 5 | slimy wounds, thick wound crusts, clearly inflamed, |
| | pink and sensitive |
| 6 | dry wounds, thick wound crusts, clearly inflamed, pink |
| | and sensitive |
| 7 | dry wounds, thick wound crusts, clearly inflamed, pink |
| | and sensitive, wound recovery from the edge |
| 8 | dry wounds, thick wound crusts, clearly inflamed, pink |
| | and sensitive, wound recovery from the edge, wound gets |
| | smaller |
| 9 | Wound is almost closed |
| 10 | Wound is completely closed |

As is shown in Table 7 below, a fast inflammation reduction was observed immediately after the treatment with Di-n-propyl disulphide, Di-n-propyl thiosulfinate, Di-n-butyl thiosulfinate, Di-methyl thiosulfonate, Di-n-propyl thiosulfonate (PTSO), and S-di-phenyl thiosulfonate. Later, the intensity of the infection was reduced and the wound healed.

By contrast, in the control treatment with clean water, no improvement of the infection was observed, and the wound became even larger. The reference example, using chlorotetracycline hydrochloride based CTC spray, did not show any effect on the wound.

In short, this Example clearly demonstrates that the claimed compounds are capable of treating a biofilm-related disorder.

TABLE 7
Wound assessment of cows at various appropriate moments after applying a control (e.g., water, Tween 80, or a reference
composition (CTC spray) or a test compound. The asterisks (*) indicate the days on which the various compositions
were applied. The percentages of organosulfur compound in the spray relates to the weight percentage.

| | | % | | | | | | | | | | |
|---|---|---|---|---|---|---|---|---|---|---|---|---|
| | CAS | compound | Number | Day | Day | Day | Day | Day | Day | Day | Day | |
| Test compound | number | in spay | of cows | 0* | 3* | 6* | 9 | 12 | 19 | 28 | 60 | Conclusion |
| Control-Water | | n.a. | 1 | 2 | 2 | 2 | 2 | 2 | 2 | 2 | 2 | No effect* |
| Control- | 9005-65-6 | 10 | 2 | 7 | 7 | 7 | 7 | 7 | 7 | 7 | 7 | No effect |
| Tween 80 | | | | 9 | 9 | 9 | 9 | 9 | 9 | 9 | 9 | |
| Di-n-propyl | 629-19-6 | 5.0 | 3 | 2 | 2 | 3 | 3 | 4 | 5 | 5 | 5 | Positive effect |
| disulfide | | | | 2 | 3 | 3 | 3 | 4 | 5 | 5 | 6 | |
| | | | | 2 | 3 | 3 | 5 | 7 | 7 | 7 | 8 | |
| Di-(n-butyl) | 629-45-8 | 12.5 | 2 | 5 | 5 | 5 | 5 | 5 | 5 | 5 | 5 | No effect |
| disulfide | | | | 6 | 6 | 5 | 5 | 5 | 5 | 5 | 5 | |
| Di-isopropyl | 4253-89-8 | 5.2 | 1 | 5 | 5 | 5 | 5 | 5 | 5 | 5 | 5 | No effect |
| disulfide | | | | | | | | | | | | |
| Di-phenyl | 882-33-7 | 12.5 | 2 | 7 | 7 | 6 | 6 | 6 | 6 | 6 | 6 | No effect |
| disulfide | | | | 7 | 7 | 7 | 7 | 7 | 6 | 6 | 6 | |
| Di-benzyl | 150-60-7 | 5 | 3 | 4 | 3 | 3 | 3 | 3 | 3 | 3 | 3 | No effect |
| disulfide | | | | 6 | 6 | 6 | 5 | 5 | 5 | 5 | 5 | |
| | | | | 6 | 5 | 5 | 6 | 5 | 5 | 5 | 5 | |
| | 5335-87-5 | 3.2 | 2 | 6 | 6 | 5 | 5 | 5 | 5 | 5 | 6 | No effect |
| Bis (4- | | | | 5 | 5 | 5 | 4 | 4 | 4 | 4 | 4 | |
| methoxyphenyl) | | | | | | | | | | | | |
| disulfide | | | | | | | | | | | | |
| Bis (4- | 405-31-2 | 3.2 | 2 | 5 | 4 | 4 | 4 | 4 | 4 | 4 | 5 | No effect |
| fluorophenyl) | | | | | | | | | | | | |
| disulfide | | | | 7 | 6 | 6 | 6 | 5 | 6 | 6 | 6 | |
| Di-p-tolyl | 103-19-5 | 3.2 | 2 | 8 | 8 | 8 | 7 | 7 | 7 | 7 | 7 | No effect |
| disulfide | | | | 4 | 4 | 3 | 3 | 2 | 2 | 3 | 3 | |
| Di-ethyl | 18542-39-7 | 20 | 1 | 8 | 8 | 7 | 7 | 7 | 7 | 6 | 7 | No effect |
| thiosulfinate | | | | | | | | | | | | |
| Di-n-propyl | 1948-52-3 | 28.5 | 1 | 2 | 2 | 3 | 4 | 7 | 7 | 7 | 7 | Positive effect |
| thiosulfinate | | | | | | | | | | | | |
| Di-n-butyl | 7559-55-9 | 20.4 | 1 | 3 | 3 | 3 | 4 | 4 | 6 | 6 | 7 | Positive effect |
| thiosulfinate | | | | | | | | | | | | |
| Di-phenyl | 1208-20-4 | | 1 | 1 | 1 | 1 | 1 | 1 | 1 | 1 | 1 | No effect |
| thiosulfinate | | | | | | | | | | | | |
| Bis (4- | 6481-72-7 | 3.2 | 2 | 6 | 6 | 5 | 5 | 5 | 5 | 5 | 4 | No/slight |
| chlorophenyl) | | | | 8 | 8 | 8 | 7 | 7 | 7 | 7 | 7 | negative effect |
| thiosulfinate | | | | | | | | | | | | |
| Bis (4- | 61169-14-0 | 3.2 | 3 | 5 | 4 | 4 | 4 | 4 | 4 | 4 | 3 | No/slight |
| fluorophenyl) | | | | 3 | 2 | 2 | 2 | 2 | 2 | 2 | 3 | negative |
| thiosulfinate | | | | 9 | 9 | 9 | 9 | 8 | 8 | 8 | 9 | effect |
| Di-methyl | 2949-92-0 | 3.5 | 1 | 3 | 3 | 4 | 4 | 7 | 8 | 9 | 10 | Positive effect |
| thiosulfonate | | | | | | | | | | | | |
| Di-n-propyl | 1113-13-9 | 3.2 | 3 | 2 | 2 | 3 | 4 | 4 | 5 | 5 | 5 | Positive effect |
| thiosulfonate | | | | 2 | 3 | 3 | 5 | 7 | 7 | ND | ND | |
| (PTSO) | | | | 2 | 2 | 3 | 3 | 4 | 4 | 5 | 6 | |
| Bis (4- | 2905-15-9 | 3.2 | 2 | 5 | 5 | 5 | 5 | 5 | 5 | 5 | 5 | Slight |
| fluorophenyl) | | | | 5 | 4 | 4 | 3 | 3 | 3 | 3 | 3 | positive effect |
| thiosulfonate | | | | | | | | | | | | |
| S-di-phenyl | 1212-08-4 | 5.9 | 2 | 2 | 4 | 5 | 5 | 6 | 7 | 7 | 4 | Positive effect |
| thiosulfonate | | | | 2 | 3 | 3 | 4 | 6 | 7 | 7 | 8 | |
| CTC spray*** | | n.v.t. | 2 | 2 | 2 | 2 | 2 | 2 | 2 | 2 | 1 | No effect |
| | | | | 2 | 2 | 2 | 2 | 2 | 2 | 2 | 2 | |

1-7. (canceled)

8. A composition comprising a compound according to Formula I: 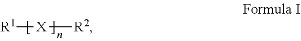
Formula I, wherein n is 2;
wherein one X is —S— and the other X is selected from the group consisting of —S(O)— and —S(O)₂—;
and R¹ and R² are independently selected from the group consisting of optionally substituted alkyl and optionally substituted aryl;
wherein the composition is a pharmaceutical composition, functional food, or a cleaning product.

9. An in vitro method for preventing or reducing biofilm formation or growth on a surface or for degrading or reducing biofilms on a surface, said method comprising applying the composition of claim 8 to the surface such as to prevent or reduce biofilm formation or growth on a surface, or such as to degrade or reduce biofilms on a surface.

10-12. (canceled)

13. An article having a surface at least partially coated with a compound according to Formula I: 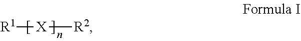
Formula I, wherein n is 2;
wherein one X is —S— and the other X is selected from the group consisting of —S(O)— and —S(O)₂—;
and R¹ and R² are independently selected from the group consisting of optionally substituted alkyl and optionally substituted aryl.

14-21. (canceled)

22. A method for treating a biofilm-related disorder or a microbial infection, said method comprising administering to an individual in need thereof a therapeutically effective amount of a compound according to Formula I, or a composition comprising a compound according to Formula I: 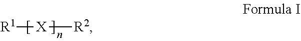
Formula I, wherein n is 2;
wherein one X is —S— and the other X is selected from the group consisting of —S(O)— and —S(O)₂—;
and R¹ and R² are independently selected from the group consisting of optionally substituted alkyl and optionally substituted aryl.

23. The method according to claim 22, wherein the biofilm-related disorder is a chronic and/or persistent infection.

24. The method according to claim 22, wherein the biofilm-related disorder is digital dermatitis, udder cleft, or a chronic wound infection.

25. The method according to claim 22, wherein the microbial infection is a bacterial or fungal infection.

26. The method according to claim 22, wherein R¹ and R² are independently selected from the group consisting of alkyl, aryl, alkylaryl, and arylalkyl, or wherein R¹ and R² are independently selected from the group consisting of C₁₋₆ alkyl, and phenyl.

27. The method according to claim 22, wherein the compound according to Formula I is not Di-n-propyl thiosulfinate or Di-n-propyl thiosulfonate,

28. The method according to claim 22, wherein when the other X is —S(O)—, R¹ and R² are independently selected from the group consisting of isopropyl, butyl, benzyl, and p-tolyl; and
wherein when the other X is —S(O)₂—, R¹ and R² are independently selected from the group consisting of methyl, phenyl, benzyl, 4-methylbenzene, and n-propyl.

29. The method according to claim 22, wherein the compound according to Formula I is selected from the group consisting of Di-n-butyl thiosulfinate, Di-methyl thiosulfonate, Di-phenyl thiosulfonate, Bis (p-tolyl) thiosulfinate, Di-isopropyl thiosulfinate, Di-benzyl thiosulfinate, Di-benzyl thiosulfonate, S-propyl-4-methylbenzene thiosulfonate, Di-n-propyl thiosulfinate, and Di-n-propyl thiosulfonate.

30. The method according to claim 22, wherein R¹ and R² are identical.

31. The method according to claim 22, wherein the compound according to Formula I is selected from the group consisting of Di-n-butyl thiosulfinate, Di-methyl thiosulfonate, Di-phenyl thiosulfonate, Bis (p-tolyl) thiosulfinate, S-propyl-4-methylbenzene thiosulfonate, Di-isopropyl thiosulfinate, Di-benzyl thiosulfinate, and Di-benzyl thiosulfonate.

32. The method according to claim 22, wherein the compound according to Formula I is selected from the group consisting of Di-n-butyl thiosulfinate, Di-methyl thiosulfonate, and Di-phenyl thiosulfonate.

33. The article according to claim 13, wherein the article is a medical device or a surgical device.
